(19) United States
(12) Patent Application Publication
(10) Pub. No.: US 2021/0230688 A1
(43) Pub. Date: Jul. 29, 2021

(54) ANALYTE DETECTION

(71) Applicant: The Regents of the University of Michigan, Ann Arbor, MI (US)

(72) Inventor: Alexander Johnson-Buck, Ann Arbor, MI (US)

(21) Appl. No.: 17/160,768

(22) Filed: Jan. 28, 2021

Related U.S. Application Data
(60) Provisional application No. 62/967,433, filed on Jan. 29, 2020.

(51) Int. Cl. C12Q 1/6874; C12Q 1/6825; G01N 21/64; G01N 1/30; C12Q 1/6837

(52) U.S. Cl. CPC C12Q 1/6874; C12Q 1/6825; C12Q 1/6837; G01N 1/30; G01N 21/6486

(57) ABSTRACT

Provided herein is technology relating to analyte detection and particularly, but not exclusively, to compositions, methods, systems, and kits for high-specificity detection of analytes using molecular probes that integrate cumulative repeated binding of probes to the same analyte molecule.

This application claims priority to U.S. provisional patent application Ser. No. 62/967,433, filed Jan. 29, 2020, which is incorporated herein by reference in its entirety.

### STATEMENT REGARDING FEDERALLY SPONSORED RESEARCH OR DEVELOPMENT

This invention was made with government support under CA204560 and CA229023 awarded by the National Institutes of Health. The government has certain rights in the invention.

### FIELD

Provided herein is technology relating to analyte detection and particularly, but not exclusively, to compositions, methods, systems, and kits for detecting, characterizing, identifying, and/or quantifying analytes (e.g., nucleic acids, proteins, or other biomolecules) using molecular probes that integrate cumulative repeated binding of probes to the same analyte molecule.

### BACKGROUND

The sensitive and specific detection of analytes (e.g., including, but not limited to, biomolecules (e.g., proteins, nucleic acids, and lipids) and small molecules (e.g., drugs, metabolites, cofactors, etc.)) plays an important role in biomedical research, in vitro diagnostics, and clinical medicine. Analyte detection is generally carried out using molecular reagents (e.g., antibodies, hybridization probes, etc.) that have a high affinity and specificity for the analyte of interest and that produce a detectable signal only in the presence of the target analyte. However, affinity probes have a finite specificity and affinity for the target analyte (see, e.g., Zhang et al. (2012) “Optimizing the specificity of nucleic acid hybridization. Nat. Chem. 4, 208-14), thus making it challenging to detect some analytes with high confidence. This is a significant problem for biomarkers of interest that are present at vanishingly small concentrations (e.g., at sub-femtomolar concentrations; see, e.g., Lee et al. (2001) “Quantitation of genomic DNA in plasma and serum samples: higher concentrations of genomic DNA found in serum than in plasma.” Transfusion (Paris) 41: 276-82; Mitchell et al. (2008) “Circulating microRNAs as stable blood-based markers for cancer detection” Proc. Natl. Acad. Sci. U.S.A 105: 10513-18; Bettegowda et al. (2014) “Detection of Circulating Tumor DNA in Early- and Late-Stage Human Malignancies” Sci. Trans. Med. 6: 224ra24-224ra24; Husain et al. (2017) “Monitoring Daily Dynamics of Early Tumor Response to Targeted Therapy by Detecting Circulating Tumor DNA in Urine” Clin. Cancer Res. 23: 4716-23).

While sensitivity can be increased by producing many copies of the analyte (e.g., by PCR and other nucleic acid amplification technologies), amplifying the signal, or using long assay and observation times to record a signal, analyte detection technologies would be improved by providing an end-point detection technology with a decreased limit of detection.

### SUMMARY

Accordingly, provided herein is a technology for detecting an analyte using molecular probes that integrate cumulative repeated binding of probes to the same analyte molecule. In some embodiments, the technology is similar to Single-Molecule Recognition with Equilibrium Poisson Sampling (SiMREPS). See, e.g., U.S. patent application Ser. No. 14/589,467; and Johnson-Buck et al. (2016) “Kinetic fingerprinting to identify and count single nucleic acids” Nature Biotechnology 33: 790, each of which is incorporated herein by reference in its entirety.

However, the technology described herein differs from SiMREPS in that the repeated binding of one probe (e.g., a “tally” probe as described herein, which is similar to a SiMREPS “query” probe) results in the accumulation of multiple labels on another probe (“integrator” probe) that records the repeated binding. The presence of the analyte is indicated by the presence of one or more integrator probes comprising multiple (e.g., more than one) labels attached to it. Accordingly, the repeated binding of tally probes to the analyte is not necessarily observed in real time in the present technology, but is detected (e.g., using an end-point measurement) by the number of labels attached to each integrator probe, e.g., either at the single-molecule level (e.g., using fluorescence microscopy) or using bulk methods (e.g., electrophoresis, chromatography, or mass spectrometry).

Accordingly, in some embodiments, the technology provides for improved (e.g., accelerated) and simpler data acquisition and/or data analysis because embodiments of the present technology can acquire data using end-point measurements rather than as a time-dependent real-time signal. However, the technology is not limited to using end-point detection and some embodiments comprise use of real-time measurements, e.g., to record a signal and/or a time-dependent signal. In some embodiments, the present technology provides a lower limit of detection than some existing (e.g., real-time (e.g., SiMREPS)) technologies, e.g., by detecting a much larger fraction of the analyte in a sample relative to existing (e.g., real-time (e.g., SiMREPS)) technologies. The technology described herein finds use in, e.g., diagnostics, research, and clinical medicine and, in some embodiments, increases the accuracy of molecular recognition in targeted therapeutics.

Accordingly, provided herein are embodiments of a composition for detecting an analyte in a sample. For example, in some embodiments, the technology provides a composition comprising an integrator probe specific for an analyte; and a tally probe specific for the analyte and comprising a label, wherein the label is irreversibly (e.g., substantially or effectively irreversibly) transferred from the tally probe to the integrator probe when the integrator probe and the tally probe are both bound to the analyte. In some embodiments, the integrator probe stably binds the analyte. In some embodiments, the tally probe transiently binds the analyte. In some embodiments, the integrator probe is configured to bind more than one label (e.g., 2, 3, 4, 5, 6, 7, 8, 9, 10, 11, 12, 13, 14, 15, 16, 17, 18, 19, or 20, or more labels). Accordingly, in some embodiments, a composition comprises an integrator probe comprising multiple labels (e.g., 2, 3, 4, 5, 6, 7, 8, 9, 10, 11, 12, 13, 14, 15, 16, 17, 18, 19, or 20, or more labels). In some embodiments, compositions as described herein further comprise an analyte. In some embodiments, the integrator probe is configured to bind a label non-covalently (e.g., irreversibly, substantially irreversibly, and/or effectively irreversibly non-covalently). In some embodiments, the integrator probe is configured to bind a label covalently (e.g., irreversibly, substantially irreversibly, and/or effectively irreversibly covalently). In some embodiments, the analyte comprises a nucleic acid, a protein, a metabolite, a small molecule, a lipid, or a sugar. In some particular embodiments, the integrator probe comprises a nucleic acid or a protein (e.g., an antibody) or both a nucleic acid and a protein. In some embodiments, the tally probe comprises a nucleic acid or a protein (e.g., an antibody) or both a nucleic acid and a protein. In some embodiments, the label is a fluorescent or luminescent label. In some embodiments, the composition further comprises a tally probe release component that converts a tally probe bound to the analyte to a dissociated tally probe. In some embodiments, the tally probe release component comprises an enzyme specific for a tally probe-analyte complex. In some embodiments, the integrator probe comprises a first functional group and the label of the tally probe comprises a second functional group that is selectively reactive towards the first functional group. In some embodiments, the integrator probe comprises a first click reactant and the label of the tally probe comprises a second click reactant. In some embodiments, the integrator probe comprises a first affinity ligand and the label of the tally probe comprises a second affinity ligand that selectively binds the first affinity ligand. In some embodiments, the integrator probe comprises a first nucleic acid sequence and the label of the tally probe comprises a second nucleic acid sequence complementary to the first nucleic acid sequence. In some embodiments, the transfer of the label from the tally probe to the integrator probe occurs via toehold-mediated strand displacement. In some embodiments, the integrator probe comprises a consensus target amino acid or nucleic acid sequence, and the label of the tally probe comprises a ligand that is enzymatically transferred to the consensus target sequence. In some embodiments, the integrator probe comprises a separate solid or liquid phase (e.g., a colloidal particle, droplet of a liquid-in-liquid emulsion, or phase of an aqueous two-phase system) into which the label of the tally probe is selectively partitioned upon binding of the tally probe to the analyte.

In a further example, in some embodiments, the technology provides a composition comprising an integrator probe that is capable of stably associating with an analyte; and a tally probe comprising a label and that is capable of directly or indirectly associating with the analyte, wherein irreversible transfer of the label from the tally probe to the integrator probe occurs more rapidly and/or more efficiently when the tally probe and the integrator probe are both associated with the analyte than when the tally probe and/or the integrator probe is/are dissociated from the analyte. In some embodiments, the composition further comprises an adaptor probe that is capable of binding to the analyte and wherein the tally probe indirectly associates with the analyte by directly associating with the adaptor probe. In some embodiments, the tally probe is specific for the analyte and directly associates with the analyte. In some embodiments, the integrator probe is specific for the analyte. In some embodiments, the tally probe transiently binds the analyte. In some embodiments, the tally probe transiently binds the adaptor probe. In some embodiments, the integrator probe is configured to bind more than one label. In some embodiments, the composition comprises an integrator probe comprising multiple labels. In some embodiments, the composition further comprises an analyte. In some embodiments, the integrator probe is configured to bind a label non-covalently. In some embodiments, the integrator probe is configured to bind a label covalently. In some embodiments, the analyte comprises a nucleic acid, a protein, a metabolite, a small molecule, a lipid, or a sugar. In some embodiments, the integrator probe comprises a separate phase of matter. In some embodiments, the separate phase of matter is a colloidal particle, liposome, micelle, or emulsified droplet. For example, in some embodiments a droplet or particle comprises the analyte (e.g., the analyte is trapped on/inside a droplet or particle) and the binding of the tally probe to the analyte results in transfer of a label to the droplet or particle. In some embodiments, the integrator probe comprises a nucleic acid or a protein. In some embodiments, the tally probe comprises a nucleic acid or a protein. In some embodiments, the label is a fluorescent or luminescent label. In some embodiments, the composition further comprises a tally probe release component that converts a tally probe bound to the analyte to a dissociated tally probe. In some embodiments, the tally probe release component comprises an enzyme specific for a tally probe-analyte complex. In some embodiments, the integrator probe comprises a first functional group and the label of the tally probe comprises a second functional group that is reactive with the first functional group. In some embodiments, the integrator probe comprises a first click reactant and the label of the tally probe comprises a second click reactant.

Furthermore, in some embodiments, the technology provides a system for detecting an analyte in a sample. For example, in some embodiments, systems comprise an integrator probe specific for an analyte; and a tally probe specific for the analyte and comprising a label, wherein the label is irreversibly (e.g., substantially or effectively irreversibly) transferred from the tally probe to the integrator probe when the integrator probe and the tally probe are both bound to the analyte. In some embodiments, the integrator probe stably binds the analyte. In some embodiments, the tally probe transiently binds the analyte. In some embodiments, the integrator probe is configured to bind more than one label (e.g., 2, 3, 4, 5, 6, 7, 8, 9, 10, 11, 12, 13, 14, 15, 16, 17, 18, 19, or 20, or more labels). In some embodiments, systems further comprise an analyte. In some embodiments, the analyte comprises a nucleic acid, a protein, a metabolite, a small molecule, a lipid, or a sugar. In some embodiments, the integrator probe comprises a nucleic acid or a protein (e.g., an antibody) or both a nucleic acid and a protein. In some embodiments, the tally probe comprises a nucleic acid or a protein (e.g., an antibody) or both a nucleic acid and a protein. In some embodiments, the label is a fluorescent or luminescent label. In some embodiments, systems further comprise a detection component configured to detect and/or quantify a signal (e.g., a fluorescence signal, a mass signal, a charge signal) produced by an integrator probe comprising multiple labels. In some embodiments, systems comprise a microprocessor (e.g., a computer). In some embodiments, systems comprise a software component configured to record a signal produced by an integrator probe, configured to detect and/or quantify the presence of an analyte in a sample using a signal produced by an integrator probe, and/or to produce an output comprising a qualitative and/or quantitative value describing the presence and/or quantity (e.g., amount and/or concentration) of an analyte in a sample. In some embodiments, the integrator probe comprises a first functional group and the label of the tally probe comprises a second functional group that is selectively reactive towards the first functional group. In some embodiments, the integrator probe comprises a first click reactant and the label of the tally probe comprises a second click reactant. In some embodiments, the integrator probe comprises a first affinity ligand and the label of the tally probe comprises a second affinity ligand that selectively binds the first affinity ligand. In some embodiments, the integrator probe comprises a first nucleic acid sequence and the label of the tally probe comprises a second nucleic acid sequence complementary to the first nucleic acid sequence. In some embodiments, the transfer of the label from the tally probe to the integrator probe occurs via toehold-mediated strand displacement. In some embodiments, the integrator probe comprises a consensus target amino acid or nucleic acid sequence and the label of the tally probe comprises a ligand that is enzymatically transferred to the consensus target sequence. In some embodiments, systems further comprise a tally probe release component that converts a tally probe bound to the analyte to a dissociated tally probe. In some embodiments, the tally probe release component comprises an enzyme specific for a tally probe-analyte complex. In some embodiments, the integrator probe comprises a separate solid or liquid phase (e.g., a colloidal particle, droplet of a liquid-in-liquid emulsion, or phase of an aqueous two-phase system) into which the label of the tally probe is selectively partitioned upon binding of the tally probe to the analyte.

In addition embodiments, the technology provides a system comprising an integrator probe that is capable of stably associating with an analyte; and a tally probe comprising a label and that is capable of directly or indirectly associating with the analyte, wherein irreversible transfer of the label from the tally probe to the integrator probe occurs more rapidly and/or more efficiently when the tally probe and the integrator probe are both associated with the analyte than when the tally probe and/or the integrator probe is/are dissociated from the analyte. In some embodiments, the system further comprises an adaptor probe that is capable of binding to the analyte and wherein the tally probe indirectly associates with the analyte by directly associating with the adaptor probe. In some embodiments, the tally probe is specific for the analyte and directly associates with the analyte. In some embodiments, the integrator probe is specific for the analyte. In some embodiments, the tally probe transiently binds the analyte. In some embodiments, the tally probe transiently binds an adaptor probe. In some embodiments, the integrator probe is configured to bind more than one label. In some embodiments, the system comprises an integrator probe comprising multiple labels. In some embodiments, the system further comprises an analyte. In some embodiments, the integrator probe is configured to bind a label non-covalently. In some embodiments, the integrator probe is configured to bind a label covalently. In some embodiments, the analyte comprises a nucleic acid, a protein, a metabolite, a small molecule, a lipid, or a sugar. In some embodiments, the integrator probe comprises a separate phase of matter. In some embodiments, the separate phase of matter is a colloidal particle, liposome, micelle, or emulsified droplet. In some embodiments, the integrator probe comprises a nucleic acid or a protein. In some embodiments, the tally probe comprises a nucleic acid or a protein. In some embodiments, the label is a fluorescent or luminescent label. In some embodiments, the system further comprise a tally probe release component that converts a tally probe bound to the analyte to a dissociated tally probe. In some embodiments, the tally probe release component comprises an enzyme specific for a tally probe-analyte complex. In some embodiments, integrator probe comprises a first functional group and the label of the tally probe comprises a second functional group that is reactive with the first functional group. In some embodiments, the integrator probe comprises a first click reactant and the label of the tally probe comprises a second click reactant. In some embodiments, systems further comprise a detection component configured to detect and/or quantify a signal produced by an integrator probe comprising multiple labels.

In some embodiments, the technology relates to methods for detecting and/or quantifying an analyte. For example, in some embodiments, methods comprise providing a sample comprising an analyte; providing an integrator probe specific for the analyte; and providing a tally probe specific for the analyte and comprising a label. In some embodiments, the label is irreversibly (e.g., substantially or effectively irreversibly) transferred from the tally probe to the integrator probe when the integrator probe and the tally probe are both bound to the analyte. In some embodiments, the integrator probe stably binds the analyte. In some embodiments, the tally probe transiently binds the analyte. In some embodiments, the integrator probe is configured to bind more than one label (e.g., 2, 3, 4, 5, 6, 7, 8, 9, 10, 11, 12, 13, 14, 15, 16, 17, 18, 19, or 20, or more labels). In some embodiments, the integrator probe is configured to bind a label non-covalently (e.g., irreversibly, substantially irreversibly, and/or effectively irreversibly non-covalently). In some embodiments, the integrator probe is configured to bind a label covalently (e.g., irreversibly, substantially irreversibly, and/or effectively irreversibly covalently). In some embodiments, the analyte comprises a nucleic acid, a protein, a metabolite, a small molecule, a lipid, or a sugar. In some embodiments, the integrator probe comprises a nucleic acid or a protein (e.g., an antibody) or both a nucleic acid and a protein. In some embodiments, the tally probe comprises a nucleic acid or a protein (e.g., an antibody) or both a nucleic acid and a protein. In some embodiments, the label is a fluorescent or luminescent label. In some embodiments, methods further comprise providing a tally probe release component that converts a tally probe bound to the analyte to a dissociated tally probe. In some embodiments, the tally probe release component comprises an enzyme specific for a tally probe-analyte complex. In some embodiments, the integrator probe comprises a first functional group and the label of the tally probe comprises a second functional group that is selectively reactive towards the first functional group. In some embodiments, the integrator probe comprises a first click reactant and the label of the tally probe comprises a second click reactant. In some embodiments, the integrator probe comprises a first affinity ligand and the label of the tally probe comprises a second affinity ligand that selectively binds the first affinity ligand. In some embodiments, the integrator probe comprises a first nucleic acid sequence and the label of the tally probe comprises a second nucleic acid sequence complementary to the first nucleic acid sequence. In some embodiments, the transfer of the label from the tally probe to the integrator probe occurs via toehold-mediated strand displacement. In some embodiments, the integrator probe comprises a consensus target amino acid or nucleic acid sequence, and the label of the tally probe comprises a ligand that is enzymatically transferred to the consensus target sequence. In some embodiments, the integrator probe comprises a separate solid or liquid phase (e.g., a colloidal particle, droplet of a liquid-in-liquid emulsion, or phase of an aqueous two-phase system) into which the label of the tally probe is selectively partitioned upon binding of the tally probe to the analyte. In some embodiments, the analyte is a biomarker for a disease. In some embodiments, the analyte is a biomarker for a cancer. In some embodiments, the sample is a biofluid. In some embodiments, the sample comprises and/or is prepared from blood, urine, mucus, saliva, semen, or tissue. In some embodiments, detecting and/or quantifying an analyte in the sample indicates that the subject has the disease. In some embodiments, methods further comprise providing a result describing the presence and/or quantity of the analyte in the sample. In some embodiments, methods further comprise providing a positive control and/or a negative control. In some embodiments, methods further comprise providing a standard curve.

Additional embodiments provide a comprising providing a sample comprising an analyte; providing an integrator probe that is capable of stably associating with an analyte; and providing a tally probe comprising a label and that is capable of directly or indirectly associating with the analyte. In some embodiments, irreversible transfer of the label from the tally probe to the integrator probe occurs more rapidly and/or more efficiently when the tally probe and the integrator probe are both associated with the analyte than when the tally probe and/or the integrator probe is/are dissociated from the analyte. In some embodiments, methods further comprise providing an adaptor probe that is capable of binding to the analyte and wherein the tally probe indirectly associates with the analyte by directly associating with the adaptor probe. In some embodiments, the tally probe is specific for the analyte and directly associates with the analyte. In some embodiments, the integrator probe is specific for the analyte. In some embodiments, the tally probe transiently binds the analyte. In some embodiments, the tally probe transiently binds the adaptor probe. In some embodiments, the integrator probe is configured to bind more than one label. In some embodiments, the integrator probe is configured to bind a label non-covalently. In some embodiments, the integrator probe is configured to bind a label covalently. In some embodiments, the analyte comprises a nucleic acid, a protein, a metabolite, a small molecule, a lipid, or a sugar. In some embodiments, the integrator probe comprises a separate phase of matter. In some embodiments, the separate phase of matter is a colloidal particle, liposome, micelle, or emulsified droplet. In some embodiments, the integrator probe comprises a nucleic acid or a protein. In some embodiments, the tally probe comprises a nucleic acid or a protein. In some embodiments, the label is a fluorescent or luminescent label. In some embodiments, methods further comprise providing a tally probe release component that converts a tally probe bound to the analyte to a dissociated tally probe. In some embodiments, the tally probe release component comprises an enzyme specific for a tally probe-analyte complex. In some embodiments, the integrator probe comprises a first functional group and the label of the tally probe comprises a second functional group that is reactive with the first functional group. In some embodiments, the integrator probe comprises a first click reactant and the label of the tally probe comprises a second click reactant. In some embodiments, the analyte is a biomarker for a disease and/or a biomarker for a cancer. In some embodiments, the sample is a biofluid. In some embodiments, the sample comprises and/or is prepared from blood, urine, mucus, saliva, semen, or tissue. In some embodiments, detecting and/or quantifying an analyte in the sample indicates that the subject has the disease. In some embodiments, methods further comprise providing a result describing the presence and/or quantity of the analyte in the sample. In some embodiments, methods further comprise providing a positive control and/or a negative control. In some embodiments, methods further comprise providing a standard curve.

In some embodiments, the technology relates to use of a composition as described herein to detect and/or quantify an analyte in a sample. In some embodiments, the technology relates to use of a system as described herein to detect and/or quantify an analyte in a sample.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings. The patent or application file contains at least one drawing executed in color. Copies of this patent or patent application publication with color drawings will be provided by the Office upon request and payment of the necessary fee.

FIG. 1 is a schematic drawing showing the principle of the technology provided herein. An integrator probe (I) binds stably to target analyte (A) and repeated binding of tally probe (T) to the same copy of A triggers the transfer of a label L (yellow star) from T to I. Initially, I comprises no labels and is designated I₀. After multiple cycles of T binding to A, I is modified by N≥2 labels and is designated I_{N}. In the presence of a spurious analyte (A*), binding of T and/or I to A* is weak and transfer of labels from T to I is thus much slower than in the presence of A. The presence of multiple (e.g., greater than 2) labels on a given copy of I provides a specific signature indicating the presence and/or quantity of A in the mixture.

FIG. 2 is a schematic drawing showing a specific exemplary embodiment of the technology using copper-free click chemistry and RNase H for detecting a nucleic acid analyte. Integrator probe I comprises multiple tetrazine (Tz, orange hexagon) moieties and binds stably to A by Watson-Crick base pairing. Tally probe T comprises a single trans-cyclooctene (TCO, green octagon) moiety and binds transiently to A because T comprises a series of RNA nucleotides complementary to A that are selectively degraded by RNase H after binding to A. Due to the high local effective concentration of the TCO and Tz labels when I and T are bound to the same copy of A, a click reaction covalently linking T and I occurs before degradation by RNase H occurs. After RNase H partially degrades the bound copy of T, the number of complementary base pairs between T and A is reduced and a new copy of T hybridizes to A. After multiple cycles of binding, click reaction, and degradation, multiple copies of partially degraded T (comprising the label L) are covalently attached to the associated copy of I, retaining a permanent memory of the occurrence of multiple binding events to the same copy of A. The presence of multiply-labeled integrator probes provides a specific signature indicating the presence and/or quantity of A in the mixture, which can be detected by a detector such as denaturing polyacrylamide electrophoresis (PAGE), fluorescence microscopy, mass spectrometry, and/or chromatography. Other embodiments of the system utilize T probes primarily comprising RNA nucleotides (lower left) and/or conjugated to affinity probes that are specific for other types of analytes (such as proteins).

FIG. 3 is a stained denaturing polyacrylamide gel showing results from an experiment in which a point mutation is detected in a 41-nucleotide mutant nucleic acid (MUT) using an integrator probe comprising up to 3 copies of TCO and a tally probe comprising methyltetrazine. The corresponding wild-type (WT) sequence, which lacks the point mutation, is not detected, showing selectivity of the assay for the point mutation.

FIG. 4 shows data from an experiment in which the presence of an analyte DNA sequence is detected by single-molecule total internal reflection fluorescence (TIRF) microscopy.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to analyte detection and particularly, but not exclusively, to compositions, methods, systems, kits, and uses of the foregoing for detecting, characterizing, identifying, and/or quantifying analytes (e.g., nucleic acids, proteins, or other biomolecules) using molecular probes (e.g., integrator probes) that integrate cumulative repeated binding of probes (e.g., tally probes) to the same analyte molecule. During the development of embodiments of the technology, experiments were conducted to develop and test a system for detecting a DNA point mutation using labels that were attached to an integrator probe via copper-free click chemistry and visualized by fluorescent staining following gel electrophoresis. In some embodiments, integrator probes comprising multiple labels are detected using single-molecule methods or other detection methods. The label can be any detectable molecular moiety such as a fluorophore, DNA sequence, affinity tag, mass tag, enzyme, hapten, protein tag, etc. and can be attached to the integrator probe covalently or non-covalently. The integrator probe may be, for example, an oligonucleotide, polypeptide, colloidal nanoparticle, or a separate phase within a system comprising multiple liquid phases, with the capability of accepting multiple (e.g., at least two) copies of the label upon repeated binding of the tally probe to the same molecule of analyte.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. For example, the term “substantially” as used herein, is a broad term and is used in its ordinary sense, including, but not limited to, being largely but not necessarily wholly that which is specified. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., an analyte). For example, when an analyte is said to be “present” in a test sample, it means the level or amount of this analyte is above a pre-determined threshold; conversely, when an analyte is said to be “absent” in a test sample, it means the level or amount of this analyte is below a pre-determined threshold. The pre-determined threshold may be the threshold for detectability associated with the particular test used to detect the analyte or any other threshold. When an analyte is “detected” in a sample it is “present” in the sample; when an analyte is “not detected” it is “absent” from the sample. Further, a sample in which an analyte is “detected” or in which the analyte is “present” is a sample that is “positive” for the analyte. A sample in which an analyte is “not detected” or in which the analyte is “absent” is a sample that is “negative” for the analyte.

As used herein, the term “detect” refers to detecting the presence or absence of an analyte or the activity or effect of an analyte or for detecting the presence or absence of a variant of an analyte

As used herein, the term “detection assay” refers to an assay for detecting the presence or absence of an analyte or the activity or effect of an analyte or for detecting the presence or absence of a variant of an analyte.

As used herein, the term “analyte” refers to a substance to be tested, assayed, detected, imaged, characterized, described, and/or quantified. Exemplary analytes include, but are not limited to, molecules, atoms, ions, biomolecules (e.g., nucleic acids (e.g., DNA, RNA) as described and as defined herein), polypeptides (e.g., peptides, proteins, glycoproteins), carbohydrates, lipids, amino acids, small molecules (drugs, bioactive agents, toxins, cofactors, metabolites), etc.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods.

As used herein, the term “sample” is used in its broadest sense. In some embodiments, a sample is or comprises an animal cell or tissue. In some embodiments, a sample includes a specimen or a culture (e.g., a microbiological culture) obtained from any source, as well as biological and environmental samples. Biological samples may be obtained from plants or animals (including humans) and encompass fluids, solids, tissues, and gases. Environmental samples include, e.g., environmental material such as surface matter, soil, water, and industrial samples, as well as samples obtained from food and dairy processing instruments, apparatuses, equipment, utensils, disposable items, and non-disposable items. These examples are not to be construed as limiting the sample types applicable to the present technology.

As used herein, a “biological sample” refers to a sample of biological tissue or fluid. For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from any of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the abovementioned samples. A biological sample may be provided by removing a sample of cells from a subject but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

As used herein, the term “biofluid” refers to a biological fluid (e.g., a body fluid, a bodily fluid). For example, in some embodiments, a biofluids is an excretion (e.g., urine, sweat, exudate (e.g., including plant exudate)) and in some embodiments a biofluid is a secretion (e.g., breast milk, bile). In some embodiments, a biofluid is obtained using a needle (e.g., blood, cerebrospinal fluid, lymph). In some embodiments, a biofluid is produced as a result of a pathological process (e.g., a blister, cyst fluid). In some embodiments, a biofluid is derived from another biofluid (e.g., plasma, serum). Exemplary biofluids include, but are not limited to, amniotic fluid, aqueous humor, vitreous humor, bile, blood, blood plasma, blood serum, breast milk, cerebrospinal fluid, cerumen (earwax), chyle, chime, endolymph, perilymph, exudates, feces, female ejaculate, gastric acid, gastric juice, lymph, mucus (e.g., nasal drainage, phlegm), pericardial fluid, peritoneal fluid, pleural fluid, pus, rheum, saliva, sebum (e.g., skin oil), serous fluid, semen, smegma, sputum, synovial fluid, sweat, tears, urine, vaginal secretion, and vomit.

The term “label” as used herein refers to any atom, molecule, molecular complex (e.g., metal chelate), or colloidal particle (e.g., quantum dot, nanoparticle, microparticle, etc.) that can be used to provide a detectable (e.g., quantifiable) effect, and that can be attached to an integrator probe and transferred to a tally probe (e.g., with or without a portion of the tally probe and/or a linker or portion thereof). Labels are compounds, structures, or elements that are amenable to at least one method of detection and/or isolation that allows for discrimination between different labels. Exemplary labels that find use with the technology provided herein include, for example, a dye, a fluorescent label, a chemiluminescent label, a quencher, a radioactive label, or combinations thereof. Labels include luminogenic and/or luminescent molecules, colored molecules (e.g., chromogens), and scintillants. Labels also include any useful linker molecule (such as biotin, avidin, digoxigenin, streptavidin, HRP, protein A, protein G, antibodies or fragments thereof, Grb2, polyhistidine, Ni²⁺, FLAG tags, myc tags), heavy metals (e.g., gold), enzymes (e.g., alkaline phosphatase, peroxidase, and luciferase), electron donors/acceptors, acridinium esters, and calorimetric substrates. It is also envisioned that a change in mass may be considered a detectable label, e.g., as finds use in surface plasmon resonance detection or mass spectrometry. Labels may provide signals detectable by fluorescence, luminescence, radioactivity, colorimetry, gravimetry, X-ray diffraction or absorption, magnetism, enzymatic activity, characteristics of mass or behavior affected by mass (e.g., mass spectrometry; fluorescence polarization), and the like. A label may be a charged moiety (positive or negative charge) or, alternatively, may be charge neutral. Labels can include or consist of nucleic acid or protein sequence, so long as the sequence comprising the label is detectable. In some embodiments, the label is a fluorescent label (e.g., a fluorophore).

As used herein the term “fluorophore” will be understood to refer to both fluorophores and luminophores and chemical agents that quench fluorescent or luminescent emissions. Further, as used herein, a “fluorophore” refers to any species possessing a fluorescent property when appropriately stimulated. The stimulation that elicits fluorescence is typically illumination; however, other types of stimulation (e.g., collisional) are also considered herein. The terms “fluorophore”, “fluor”, “fluorescent moiety”, “fluorescent dye”, and “fluorescent group” are used interchangeably. In some embodiments, a fluorescent label comprises a fluorophore as described below in the section entitled “Fluorescent labels”.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino, locked nucleic acid (LNA), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand.

The term “nucleotide analog” as used herein refers to modified or non-naturally occurring nucleotides including but not limited to analogs that have altered stacking interactions such as 7-deaza purines (e.g., 7-deaza-dATP and 7-deaza-dGTP); base analogs with alternative hydrogen bonding configurations (e.g., such as Iso-C and Iso-G and other non-standard base pairs, e.g., as described in U.S. Pat. No. 6,001,983, which is incorporated by reference); non-hydrogen bonding analogs (e.g., non-polar, aromatic nucleoside analogs such as 2,4-difluorotoluene, e.g., as described by Schweitzer and Kool, J. Org. Chem., 1994, 59, 7238-7242, Schweitzer and Kool, J. Am. Chem. Soc., 1995, 117, 1863-1872; each of which is incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152, each of which is incorporated by reference). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

“Peptide nucleic acid” means a DNA mimic that incorporates a peptide-like polyamide backbone.

As used herein, the terms “complementary” or “complementarity” are used in reference to polynucleotides (e.g., a sequence of nucleotides such as an oligonucleotide tally probe, oligonucleotide integrator probe, or a target analyte that is a nucleic acid) related by the base-pairing rules. For example, for the sequence “5′-A-G-T-3′” is complementary to the sequence “3′-T-C-A-5′.” Complementarity may be “partial”, in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions and in detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary” and “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide(s), base composition of the oligonucleotide(s), sequence of the oligonucleotide(s), ionic strength of a composition comprising the oligonucleotide(s), and incidence of mismatched base pairs between two oligonucleotides.

Thus, in some embodiments, “complementary” refers to a first nucleic acid sequence that is at least 60%, 65%, 70%, 75%, 80%, 85%, 90%, 95%, 97%, 98%, or 99% identical to the complement of a second nucleic acid sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

As used herein, the term “mismatch” refers to a nucleobase of a first nucleic acid that is not capable of pairing with a nucleobase at a corresponding position of a second nucleic acid.

As used herein, the term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified”, “mutant”, “variant”, or “polymorphic” refers to a gene or gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product. Thus, the terms “variant” and “mutant” when used in reference to a nucleotide sequence refer to a nucleic acid sequence that differs by one or more nucleotides from another, usually related nucleotide acid sequence. A “variation” is a difference between two different nucleotide sequences; in some embodiments, one sequence is a “reference sequence”.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, e.g., a nucleic acid having a complementary nucleotide sequence. The ability of two polymers of nucleic acid comprising complementary sequences to find each other and anneal through base pairing interaction is a well-recognized phenomenon. The initial observations of the “hybridization” process by Marmur and Lane, Proc. Natl. Acad. Sci. USA 46: 453 (1960) and Doty et al., Proc. Natl. Acad. Sci. USA 46:461 (1960), each of which is incorporated herein by reference, have been followed by the refinement of this process into an essential tool of modern biology.

As used herein, the term “toehold” refers to a short (e.g., comprising 1-10 (e.g., 1, 2, 3, 4, 5, 6, 7, 8, 9, or 10 nt)) single-stranded nucleic acid extension that is adjacent to a nucleic acid duplex and that accelerates the binding of a third nucleic acid to one of the strands of the duplex and that displaces one strand of the original duplex. Thus, a “toehold” may provide a nucleation site of a nucleic acid that is configured to initiate hybridization of a complementary nucleic acid sequence. In some embodiments, the rate constants of hybridization between complementary oligonucleotide sequences can be manipulated over a range of approximately 1 million-fold using a phenomenon known as toehold-mediated strand displacement (see, e.g., Zhang & Winfree (2009) “Control of DNA strand displacement kinetics using toehold exchange.” J. Am. Chem. Soc. 131: 17303-14, incorporated herein by reference).

As used herein, the term “displacement” encompasses both complete displacement and at least partial displacement. As will be appreciated by one of skill in the art, partial displacement may be sufficient for various embodiments herein, and/or could occur before complete displacement occurs. Complete or partial displacement will be adequate for the function to be achieved. Complete or partial displacement can each be specified as desired by the term “complete” or “partial”.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41*(% G+C), when a nucleic acid is in aqueous solution at 1 M NaCl (see, e.g., Anderson and Young, “Quantitative Filter Hybridization” in Nucleic Acid Hybridization (1985), incorporated herein by reference. Other references (e.g., Allawi and SantaLucia, Biochemistry 36: 10581-94 (1997), incorporated herein by reference) include more sophisticated computations which account for structural, environmental, and sequence characteristics to calculate Tₘ. For example, in some embodiments these computations provide an improved estimate of Tₘ for short nucleic acid probes and targets (e.g., as used in the examples).

The terms “protein” and “polypeptide” refer to compounds comprising amino acids joined via peptide bonds and are used interchangeably. A “protein” or “polypeptide” encoded by a gene is not limited to the amino acid sequence encoded by the gene but includes post-translational modifications of the protein. Where the term “amino acid sequence” is recited herein to refer to an amino acid sequence of a protein molecule, “amino acid sequence” and like terms such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. Conventional one-letter and three-letter amino acid codes are used herein as follows—Alanine: Ala, A; Arginine: Arg, R; Asparagine: Asn, N; Aspartate: Asp, D; Cysteine: Cys, C; Glutamate: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; Valine: Val, V. As used herein, the codes Xaa and X refer to any amino acid.

The terms “variant” and “mutant” when used in reference to a polypeptide refer to an amino acid sequence that differs by one or more amino acids from another, usually related polypeptide.

As used herein, a “stable interaction” or referring to a “stably bound” interaction refers to an association of two entities or components that is relatively persistent under the thermodynamic equilibrium conditions of the interaction. In some embodiments, a “stable interaction” is an interaction between two components having a K_{D} that is smaller than approximately 10⁻⁹ M or, in some embodiments, a K_{D} that is smaller than 10⁻⁸ M. In some embodiments, a “stable interaction” has a dissociation rate constant km that is smaller than 1 per hour or, in some embodiments, a dissociation rate constant k_{off} that is smaller than 1 per minute. In some embodiments, a “stable interaction” is defined as not being a “transient interaction” and a “transient interaction” is defined as not being a “stable interaction”. In some embodiments, a “stable interaction” includes interactions mediated by covalent bonds and other interactions that are not typically described by a K_{D} value but that involve an average association lifetime between two entities that is longer than approximately 1 minute (e.g., 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 seconds) per each interaction.

In some embodiments, the distinction between a “stable interaction” and a “transient interaction” is determined by a cutoff value of K_{D} and/or k_{off} and/or another kinetic or thermodynamic value describing the associations, wherein the cutoff is used to discriminate between stable and transient interactions that might otherwise be characterized differently if described in absolute terms of a K_{D} and/or k_{off} another kinetic or thermodynamic value describing the associations. For example, a “stable interaction” characterized by a K_{D} value might also be characterized as a “transient interaction” in the context of another interaction that is even more stable. One of skill in the art would understand other relative comparisons of stable and transient interactions, e.g., that a “transient interaction” characterized by a K_{D} value might also be characterized as a “stable interaction” in the context of another interaction that is even more transient (less stable).

As used herein, the terms “stable interaction”, “stable binding”, and “stable association” are used interchangeably. As used herein, the terms “transient interaction”, “transient binding”, and “transient association” are used interchangeably.

The terms “specific binding” or “specifically binding” when used in reference to the interaction of two components A and B that associate with one another refers to an association of A and B having a K_{D} that is smaller than the K_{D} for the interaction of A or B with other similar components in the solution, e.g., at least one other molecular species in the solution that is not A or B.

As used herein, the term “affinity” refers to the strength of interaction (e.g., binding) of one entity (e.g., molecule) with another entity (e.g., molecule), e.g., an antibody with an antigen. In some embodiments, affinity depends on the closeness of stereochemical fit between entities, on the size of the area of contact between them, on the distribution of charged and hydrophobic groups, etc.

As used herein, the term “irreversible interaction” refers to an interaction (e.g., association, binding, etc.) having a dissociation half-life longer than the incubation time, e.g., in some embodiments, a time that is 1 to 10 minutes (e.g., 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 seconds, or longer).

As used herein, the term “sensitivity” refers to the probability that an assay gives a positive result for the analyte when the sample comprises the analyte. Sensitivity is calculated as the number of true positive results divided by the sum of the true positives and false negatives. Sensitivity is a measure of how well an assay detects an analyte.

As used herein, the term “specificity” refers to the probability that an assay gives a negative result when the sample does not comprise the analyte. Specificity is calculated as the number of true negative results divided by the sum of the true negatives and false positives. Specificity is a measure of how well a method of the present invention excludes samples that do not comprise an analyte from those that do comprise the analyte.

As used herein, the “equilibrium constant” (K_{eq}), the “equilibrium association constant” (Kₐ), and “association binding constant” (or “binding constant” (K_{B})) are used interchangeably for the following binding reaction of A and B at equilibrium:

A+B [symbol] AB

where A and B are two entities that associate with each other (e.g., tally probe and analyte, integrator probe and analyte, label and integrator probe, label and tally probe, etc.) and K_{eq}=[AB]/([A]×[B]). The dissociation constant K_{D}=1/K_{B}. The K_{D} is a useful way to describe the affinity of a one binding partner A for a partner B with which it associates, e.g., the number K_{D} represents the concentration of A or B that is required to yield a significant amount of AB. K_{eq}=k_{off}; K_{D}=k_{off}/kₒₙ. Accordingly, the dissociation constant, K_{D}, and the association constant, KA, are quantitative measures of affinity. At equilibrium, A and B are in equilibrium with A-B complex, and the rate constants, kₐ and k_{d}, quantify the rates of the individual forward and backward reactions of the equilibrium state:

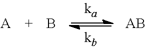

At equilibrium, kₐ [A][B]=k_{d} [AB]. The dissociation constant, K_{D}, is given by K_{D}=k_{d}/kₐ=[A][B]/[AB]. K_{D} has units of concentration, e.g., M, mM, μM, nM, pM, etc. When comparing affinities expressed as K_{D}, a greater affinity is indicated by a lower value. The association constant, K_{A}, is given by K_{A}=K_{A}/K_{D}=[AB]/[A][B]. KA has units of inverse concentration, most typically M⁻¹, mM⁻¹, μM⁻¹, nM⁻¹, pM⁻¹, etc.

As used herein, “moiety” refers to one of two or more parts into which something may be divided, such as, for example, the various parts of an oligonucleotide, a molecule, a chemical group, a domain, a probe, an “R” group, a polypeptide, etc.

In some embodiments, the technology comprises an antibody component or moiety, e.g., an antibody or fragments or derivatives thereof. As used herein, an “antibody”, also known as an “immunoglobulin” (e.g., IgG, IgM, IgA, IgD, IgE), comprises two heavy chains linked to each other by disulfide bonds and two light chains, each of which is linked to a heavy chain by a disulfide bond. The specificity of an antibody resides in the structural complementarity between the antigen combining site of the antibody (or paratope) and the antigen determinant (or epitope). Antigen combining sites are made up of residues that are primarily from the hypervariable or complementarity determining regions (CDRs). Occasionally, residues from non-hypervariable or framework regions influence the overall domain structure and hence the combining site. Some embodiments comprise a fragment of an antibody, e.g., any protein or polypeptide-containing molecule that comprises at least a portion of an immunoglobulin molecule such as to permit specific interaction between said molecule and an antigen. The portion of an immunoglobulin molecule may include, but is not limited to, at least one complementarity determining region (CDR) of a heavy or light chain or a ligand binding portion thereof, a heavy chain or light chain variable region, a heavy chain or light chain constant region, a framework region, or any portion thereof. Such fragments may be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. The various portions of antibodies can be joined together chemically by conventional techniques or can be prepared as a contiguous protein using genetic engineering techniques.

Fragments of antibodies include, but are not limited to, Fab (e.g., by papain digestion), F(ab′)2 (e.g., by pepsin digestion), Fab′ (e.g., by pepsin digestion and partial reduction) and Fv or scFv (e.g., by molecular biology techniques) fragments. A Fab fragment can be obtained by treating an antibody with the protease papaine. Also, the Fab may be produced by inserting DNA encoding a Fab of the antibody into a vector for prokaryotic expression system or for eukaryotic expression system and introducing the vector into a prokaryote or eukaryote to express the Fab. A F(ab′)2 may be obtained by treating an antibody with the protease pepsin. Also, the F(ab′)2 can be produced by binding a Fab′ via a thioether bond or a disulfide bond. A Fab may be obtained by treating F(ab′)₂ with a reducing agent, e.g., dithiothreitol. Also, a Fab′ can be produced by inserting DNA encoding a Fab′ fragment of the antibody into an expression vector for a prokaryote or an expression vector for a eukaryote and introducing the vector into a prokaryote or eukaryote for its expression. A Fv fragment may be produced by restricted cleavage by pepsin, e.g., at 4° C. and pH 4.0. (a method called “cold pepsin digestion”). The Fv fragment consists of the heavy chain variable domain (V_{H}) and the light chain variable domain (V_{L}) held together by strong noncovalent interaction. A scFv fragment may be produced by obtaining cDNA encoding the V_{H} and V_{L} domains as previously described, constructing DNA encoding scFv, inserting the DNA into an expression vector for prokaryote or an expression vector for eukaryote, and then introducing the expression vector into a prokaryote or eukaryote to express the scFv.

In general, antibodies can usually be raised to any antigen, using the many conventional techniques now well known in the art.

As used herein, the term “conjugated” refers to when one molecule or agent is physically or chemically coupled or adhered to another molecule or agent. Examples of conjugation include covalent linkage and electrostatic complexation. The terms “complexed,” “complexed with,” and “conjugated” are used interchangeably herein.

As used herein, the term “incubation temperature” refers to a temperature at which a composition comprising an integrator probe, a tally probe, and a sample is incubated (e.g., held or maintained constant or substantially constant or effectively constant) prior to observing the composition to detect, characterize, and/or quantify the analyte, if present, in the sample (e.g., to record data indicating the presence, absence, and/or quantity of analyte, if present, in the sample). In some embodiments, the “incubation temperature” is controlled using active heating and cooling (e.g., a heat pump, Peltier device, water bath, or other technology known in the art to maintain the temperature of a sample, e.g., within 0.1 to 0.5 to 1.0° C. of a set temperature). In some embodiments, the incubation temperature is 10 to 110° C. (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110° C.).

As used herein, the term “incubation time” refers to a length of time for which a composition comprising an integrator probe, a tally probe, and a sample is incubated (e.g., held or maintained constant or substantially constant or effectively constant) at the incubation temperature prior to observing the composition to detect, characterize, and/or quantify the analyte, if present, in the sample (e.g., to record data indicating the presence, absence, and/or quantity of analyte, if present, in the sample). The incubation time is sufficient for multiple cycles of a tally probe binding to an analyte molecule and label transfer from a tally probe to an integrator probe to produce one or more copies of the integrator probe that is/are labeled with multiple copies of the label. In some embodiments, the “incubation time” refers to an amount of time that is tens to hundreds to thousands of seconds, e.g., 5, 10, 15, 20, 25, 30, 35, 40, 45, 50, 55, or 60 seconds; e.g., 5, 10, 15, 20, 25, 30, 35, 40, 45, 50, 55, or 60 minutes; e.g., 1, 1.5, 2, 2.5, or 3 hours. In some embodiments, the “incubation time” refers to an amount of time that is approximately 1 to 10 seconds to 1 to 10 minutes (e.g., approximately 1 to 100 seconds, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 seconds, e.g., 1 to 100 minutes, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 minutes. In some embodiments, the incubation time comprises multiple shorter incubation times (e.g., a first incubation time, a second incubation time, a third incubation time, etc.) at different incubation temperatures (e.g., a first incubation temperature, a second incubation temperature, a third incubation temperature, etc.)

As used herein, the term “integrator probe” refers to any entity (e.g., molecule, biomolecule, colloidal particle, liquid phase, etc.) that binds stably to an analyte and that can irreversibly (e.g., substantially irreversibly and/or effectively irreversibly) accept (e.g., stably bind covalently or stably bind non-covalently) multiple labels from one or more tally probes. In some embodiments, the integrator probe binds an analyte with high thermodynamic stability (e.g., with a melting temperature more than 10 degrees C. above the incubation temperature or an average lifetime longer than the incubation time). In some embodiments, the integrator probe is covalently linked to a plurality of labels. In some embodiments, the integrator probe is indirectly and/or non-covalently linked and/or associated with a plurality of labels. In some embodiments, the integrator probe is a protein that recognizes an analyte. In some embodiments, the integrator probe is a nucleic acid (e.g., an oligonucleotide) that recognizes an analyte. For example, in some embodiments the integrator probe comprises, e.g., DNA, RNA, a nucleic acid comprising DNA and RNA, a nucleic acid comprising modified bases and/or modified linkages between bases such as, e.g., a nucleic acid as described herein. In some embodiments, the nucleic acid integrator probe comprises a nucleic acid aptamer. In some embodiments, the integrator probe comprises more than one type of molecule (e.g., more than one of a protein, a nucleic acid, a chemical linker, or a chemical moiety). For example, in some embodiments, the integrator probe comprises a conjugate comprising a protein and a nucleic acid (e.g., a protein-nucleic acid conjugate in which a nucleic acid (e.g., an oligonucleotide) is covalently linked to a polypeptide (e.g., a protein and/or a peptide) to provide a chimeric molecule. In some embodiments, the integrator probe comprises an antibody (e.g., a monoclonal antibody) or antibody fragment. In some embodiments, the integrator probe comprises a nanobody, a DNA-binding protein, or a DNA-binding protein domain. In some embodiments, the integrator probe is a small organic molecule (e.g., a molecule having a molecular weight that is less than approximately 2000 daltons, e.g., less than 2100, 2050, 2000, 1950, 1900, 1850, 1800, 1750, 1700, 1650, 1600, 1550, 1500 daltons, or less). In some embodiments, the integrator probe is labeled prior to associating with an analyte (e.g., in some embodiments, the integrator probe is labeled with a first label and can irreversibly accept (e.g., stably bind covalently or non-covalently) multiple second labels from one or more tally probes). In some embodiments in which the analyte comprises a carbohydrate or polysaccharide, the integrator probe comprises a carbohydrate-binding protein such as a lectin or a carbohydrate-binding antibody. In some embodiments, the integrator probe is engineered to strengthen its binding relative to the non-engineered version, e.g., to provide a binding and/or association with an analyte that is more thermodynamically stable than the non-engineered version. In addition to integrator probes comprising antibodies, embodiments comprise target analytes and integrator probes that are proteins, e.g., where one binding partner is the integrator probe and the other binding partner is the analyte being measured. Embodiments comprise the use of aptamers binding any ligand, lectins binding glycosylated proteins, proteins or other molecules binding lipids, etc. The technology comprises the use of any binding pair with stable binding behavior suitable for detection as described herein.

As used herein, the term “tally probe” refers to any entity (e.g., molecule, biomolecule, etc.) that binds transiently to an analyte and that comprises a label that can be irreversibly (e.g., substantially irreversibly or effectively irreversibly) transferred from the tally probe to an integrator probe. In some embodiments, the tally probe has a dissociation constant (K_{D}) for the analyte of larger than approximately 0.1 nanomolar (e.g., greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more nanomolar) under the assay conditions (e.g., incubation temperature, probe concentrations, pH, salt concentration, and/or the presence of a tally probe release component). The technology contemplates a broad range of analyte types, tally probe types, and associated types and/or strengths of transient interactions between the tally probe and the analyte. Accordingly, in some embodiments, the tally probe has a dissociation constant (K_{D}) for the analyte that is in the range of approximately 0.1 nm to approximately 100 nm (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6.0, 7.0, 8.0, 9.0, 10, 15, 20, 25, 30, 35, 40, 45, 50, 55, 60, 65, 70, 75, 80, 85, 90, 95, or 100 nm) under the assay conditions (e.g., incubation temperature, probe concentrations, pH, salt concentration, and/or the presence of a tally probe release component). In some embodiments, e.g., in which a tally probe has more than one form (e.g., is converted from a first form (e.g., a form bound to an analyte) to another form (e.g., a form that dissociates from the analyte)), e.g., by a tally probe release component, the first form of the tally probe has a first dissociation constant (K_{D}) for the analyte that is in the range of approximately 1 to approximately 100 picomolar (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6.0, 7.0, 8.0, 9.0, 10, 15, 20, 25, 30, 35, 40, 45, 50, 55, 60, 65, 70, 75, 80, 85, 90, 95, or 100 nm) under the assay conditions (e.g., incubation temperature, probe concentrations, pH, salt concentration, and/or the presence of a tally probe release component) and the second form of the tally probe has a second dissociation constant (K_{D}) for the analyte that is in the range of approximately 10 nm to approximately 1000 nm (e.g., 10, 20, 30, 40, 50, 60, 70, 80, 90, 100, 200, 300, 400, 500, 600, 700, 800, 900, or 1000 nm) under the assay conditions (e.g., incubation temperature, probe concentrations, pH, salt concentration, and/or the presence of a tally probe release component).

In some embodiments, the tally probe has a binding rate constant and/or a dissociation rate constant for the analyte that is larger than approximately 0.1 min-1 (e.g., greater than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more min⁻¹) under the assay conditions (e.g., incubation temperature, probe concentrations, pH, salt concentration, and/or the presence of a tally probe release component). The technology contemplates a broad range of analyte types, tally probe types, and associated types and/or strengths of transient interactions between the tally probe and the analyte. Accordingly, in some embodiments, the tally probe has a binding rate constant and/or a dissociation rate constant for the analyte that is in the range of approximately 0.1 min-1 to 10 min-1 (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 min⁻¹) under the assay conditions (e.g., incubation temperature, probe concentrations, pH, salt concentration, and/or the presence of a tally probe release component). In some embodiments, e.g., in which a tally probe has more than one form (e.g., is converted from a first form (e.g., a form bound to an analyte) to another form (e.g., a form that dissociates from the analyte)), e.g., by a tally probe release component, the first form of the tally probe has a first dissociation rate constant for the analyte that is approximately less than 0.1 min⁻¹ (e.g., less than 0.10, 0.09, 0.08, 0.07, 0.06, 0.05, 0.04, 0.03, 0.02, 0.01, 0.010, 0.009, 0.008, 0.007, 0.006, 0.005, 0.004, 0.003, 0.002, or 0.001 min⁻¹) under the assay conditions (e.g., incubation temperature, probe concentrations, pH, salt concentration, and/or the presence of a tally probe release component) and the second form of the tally probe has a second dissociation rate constant for the analyte that is in the range of approximately 0.1 min-1 to approximately 10 min⁻¹ (e.g., 10.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 min-) under the assay conditions (e.g., incubation temperature, probe concentrations, pH, salt concentration, and/or the presence of a tally probe release component). In some embodiments, the binding rate constant of the tally probe with the analyte is at least 105 to 10⁶ M₋₁ s⁻¹.

In some embodiments, the transient binding of a tally probe results from converting a stably bound tally probe into a transiently bound tally probe that dissociates from the analyte, e.g., by an enzymatic reaction or change in conformation that converts a tally probe stably bound to an analyte into a transiently bound tally probe that dissociates from the analyte. In some embodiments, the tally probe is covalently linked to a label that can be transferred to an integrator probe. In some embodiments, the tally probe is indirectly and/or non-covalently linked and/or associated with a label that can be transferred to an integrator probe. In some embodiments, the tally probe is a protein that recognizes an analyte. In some embodiments, the tally probe is a nucleic acid (e.g., an oligonucleotide) that recognizes an analyte. For example, in some embodiments the tally probe comprises, e.g., DNA, RNA, a nucleic acid comprising DNA and RNA, a nucleic acid comprising modified bases and/or modified linkages between bases such as, e.g., a nucleic acid as described herein. In some embodiments, a tally probe comprises a first portion comprising DNA nucleotides and a second portion comprising RNA nucleotides. In some embodiments, a tally probe comprises DNA nucleotides and RNA nucleotides in a ratio of approximately 10:1, 5:1, 2:1, 1:1, 1:2, 1:5, or 1:10. In some embodiments, the nucleic acid tally probe comprises a nucleic acid aptamer. In some embodiments, the tally probe comprises more than one type of molecule (e.g., more than one of a protein, a nucleic acid, a chemical linker, or a chemical moiety). In some embodiments, the tally probe comprises an antibody (e.g., a monoclonal antibody) or antibody fragment. In some embodiments, the tally probe comprises a low-affinity antibody (e.g., monoclonal antibody) or antibody fragment. In some embodiments, the tally probe comprises a nanobody, a DNA-binding protein, or a DNA-binding protein domain. In some embodiments, the tally probe is a small organic molecule (e.g., a molecule having a molecular weight that is less than approximately 2000 daltons, e.g., less than 2100, 2050, 2000, 1950, 1900, 1850, 1800, 1750, 1700, 1650, 1600, 1550, 1500 daltons, or less). In some embodiments in which the analyte comprises a carbohydrate or polysaccharide, the tally probe comprises a carbohydrate-binding protein such as a lectin or a carbohydrate-binding antibody. In some embodiments, the tally probe is engineered to weaken its binding relative to the non-engineered version, e.g., to provide a binding and/or association with an analyte that is less thermodynamically stable and/or more transient than the non-engineered version. In addition to tally probes comprising antibodies, embodiments comprise target analytes and tally probes that are proteins, e.g., where one binding partner is the tally probe and the other binding partner is the analyte being measured. Embodiments comprise the use of aptamers binding any ligand, lectins binding glycosylated proteins, proteins or other molecules binding lipids, etc. The technology comprises the use of any binding pair with transient binding behavior suitable for detection as described herein.

As used herein, the term “tally probe release component” refers to an entity (e.g., molecule, biomolecule (e.g., enzyme)) that promotes the dissociation of a tally probe from a tally probe-analyte complex, e.g., after transfer of a label from the tally probe to an integrator probe. That is, the tally probe release component converts a tally probe associated with an analyte to a tally probe that is dissociated from the analyte or that rapidly dissociates from the analyte (e.g., with a dissociation rate constant 0.1 min⁻¹ to approximately 10 min⁻¹ (e.g., 10.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 min-)) under the assay conditions (e.g., incubation temperature, probe concentrations, pH, and/or salt concentration). In some embodiments, a first portion of the tally probe (e.g., a portion comprising a label) remains associated with the integrator probe and a second portion of the tally probe dissociates from the analyte. In some embodiments, the tally probe release component is an enzyme (e.g., an RNase H) that degrades a portion of the tally probe (e.g., a portion of a tally probe comprising RNA) when it is bound to an analyte (e.g., a DNA analyte).

As used herein, the term “click chemistry” or “click reaction” refers to a chemical reaction that has a desirable chemical yield, yields a physiologically stable product, and that exhibits a large thermodynamic driving force that favors a “spring-loaded” reaction that yields a single product. See, e.g., Huisgen (1961) “Centenary Lecture—1,3-Dipolar Cycloadditions”, Proceedings of the Chemical Society of London 357; Kolb, Finn, Sharpless (2001) “Click Chemistry: Diverse Chemical Function from a Few Good Reactions”, Angewandte Chemie International Edition 40(11): 2004-2021, each of which is incorporated herein by reference. Click chemistry may comprise Diels-Alder reactions, thiol-yne reactions, azide-alkyne reactions, tetrazine-trans-cyclooctene reactions, a combination thereof, or other reactions that form a single physiologically stable product (e.g., a covalent bond) with a desirable chemical yield and large thermodynamic driving force (e.g., in a “spring-loaded” reaction). For example, a foundational click reaction is the reaction of an alkyne with an azide group (e.g., Na, e.g., N═N═N) in a copper (I)-catalyzed azide-alkyne cycloaddition (“CuAAC”) reaction to form two new covalent bonds between azide nitrogens and alkyl carbons. The covalent bonds form a chemical link (e.g., comprising a five-membered triazole ring) between a first component and a second component that comprised the azide and the alkyne moieties before linkage. In some embodiments, the reaction is performed in a milieu comprising a copper-based catalyst such as Cu/Cu(OAc)₂, a tertiary amine such as tris-(benzyltriazolylmethyl)amine (TBTA), and/or tetrahydrofuran and acetonitrile (THF/MeCN). In some embodiments, a click reaction is a copper-free click reaction, e.g., a click reaction between a tetrazine moiety (e.g., a six-membered aromatic ring comprising four nitrogen atoms (C₂H₂N₄), e.g., 1,2,3,4-tetrazine, 1,2,3,5-tetrazine, or 1,2,4,5-tetrazine) and a trans-cyclooctene moiety (e.g., (CH₂)₆(CH)₂). See, e.g., Jewett and Bertozzi (2010) “Cu-free click cycloaddition reactions in chemical biology” Chem Soc Rev 39: 1272-79; Freidel (2016) “Chemical tags for site-specific fluorescent labeling of biomolecules” Amino Acids 48: 1357-72; and Kim and Koo (2019) “Biomedical applications of copper-free click chemistry: in vitro, in vivo, and ex vivo” Chem Sci 10: 7835-51, each of which is incorporated herein by reference. As used herein, the two moieties that react in a click reaction to form a product are called a “first click reactant” and a “second click reactant”. As used herein the “first click reactant” and the “second click reactant” form a “click product” in a click reaction. In some embodiments, a “click catalyst” promotes the formation of a click product from the first click reactant and the second click reactant (e.g., by lowering the activation energy of the click reaction).

### DESCRIPTION

Although the disclosure herein refers to certain illustrated embodiments, it is to be understood that these embodiments are presented by way of example and not by way of limitation.

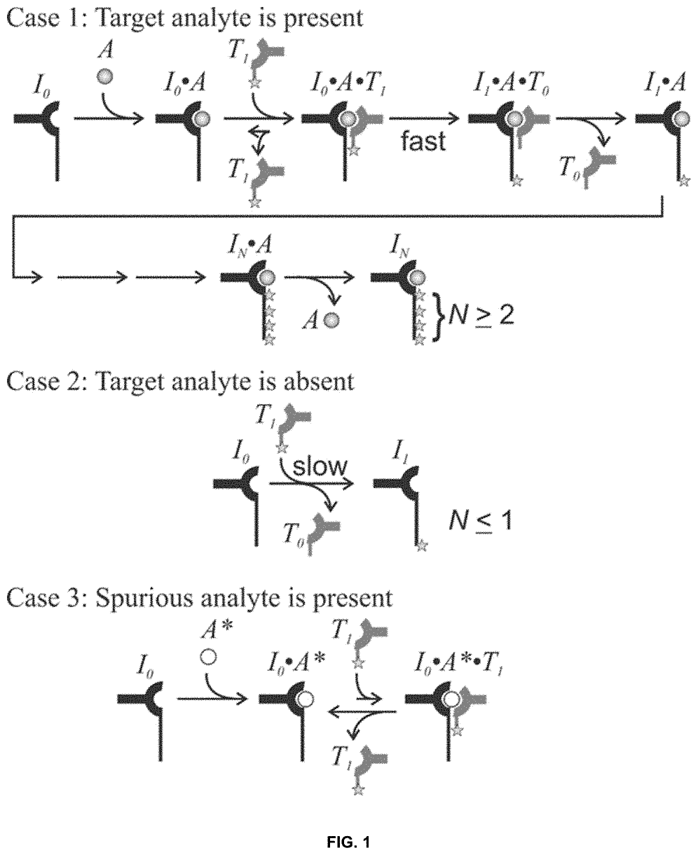

Compositions for Analyte Detection In some embodiments, the technology provides compositions for detecting an analyte using an integrator probe and a tally probe. The integrator probe (I) binds stably (e.g., thermodynamically stable) to an analyte (A) and is capable of irreversibly and/or substantially irreversibly and/or effectively irreversibly binding (e.g., covalently or non-covalently) multiple copies of a label moiety (L). The tally probe (T) binds transiently to the analyte (A) and comprises a label moiety (U. Upon binding of the tally probe (T) to the analyte (A), the tally probe (T) irreversibly or substantially irreversibly or effectively irreversibly transfers a label moiety (L) to an integrator probe (I) (e.g., a proximal copy of the integrator probe (I)). That is, a tally probe (T) bound to the analyte (A) transfers a label moiety (L) to the integrator probe (I) bound to the same analyte molecule (A) with a high probability. The integrator probe (I) and the tally probe (T) are incubated with a composition comprising an analyte (A) for a sufficient length of time (e.g., an incubation time) to provide multiple cycles of tally probe (T) binding to each analyte (A) molecule present, thus producing one or more copies of the integrator probe (I) that is/are labeled with multiple copies of the label moiety (L). See, e.g., FIG. 1.

Thus, an integrator probe irreversibly binds multiple (e.g., at least 2) label moieties with high probability when (e.g., if and only if) the integrator probe is bound to the analyte (FIG. 1). In the absence of analyte (A), or in the presence of a spurious analyte (A*), the much lower bulk concentrations of integrator probe and tally probe provide that transfer of a label moiety to an integrator probe is an exceedingly rare event, and the vast majority of integrator probes comprise a small number of label moieties (e.g., 0 or 1) (FIG. 1). In other words, the presence of the analyte greatly accelerates the generation of multiply labeled integrator probes, which are generated only very slowly in the absence of the analyte. The analyte is detected by the presence of one or more copies of the integrator probe bearing at least N (e.g., at least 2) copies of the label. Detection is accomplished by any method that can distinguish between integrator probes comprising multiple label moieties from integrator probes comprising no or fewer label moieties.

As shown in FIG. 1, An integrator probe I binds stably to analyte A and repeated binding of tally probe T to the same copy of A triggers the transfer of a label L (star) from T to I. This transfer of the label is rapid due to the high local effective concentrations of I and T caused by binding to the same copy of A. Initially, I comprises no labels from the binding of T, and is designated I₀. After multiple cycles of T binding, I is modified by N≥2 labels, and is designated I_{N}. I thus retains a memory of how many cycles of T binding have occurred to the associated copy of A. In the absence of analyte A (FIG. 1, Case 2), transfer of labels from T to I is slow because T and I are not colocalized (e.g., there is no locally high concentration of T and I and thus no enhancement of label transfer T to I). In the presence of a spurious analyte A* (FIG. 1, Case 3), binding of T and/or I to A* is weak, and the equilibrium favors states in which the two probes (I and T) are not simultaneously bound to the same copy of A*. The transfer of labels from T to I is thus much slower in the presence of A* than in the presence of A. The presence of multiple (e.g., more than 2) labels on a given copy of I thus provides a specific signature indicating the presence of A in the mixture.

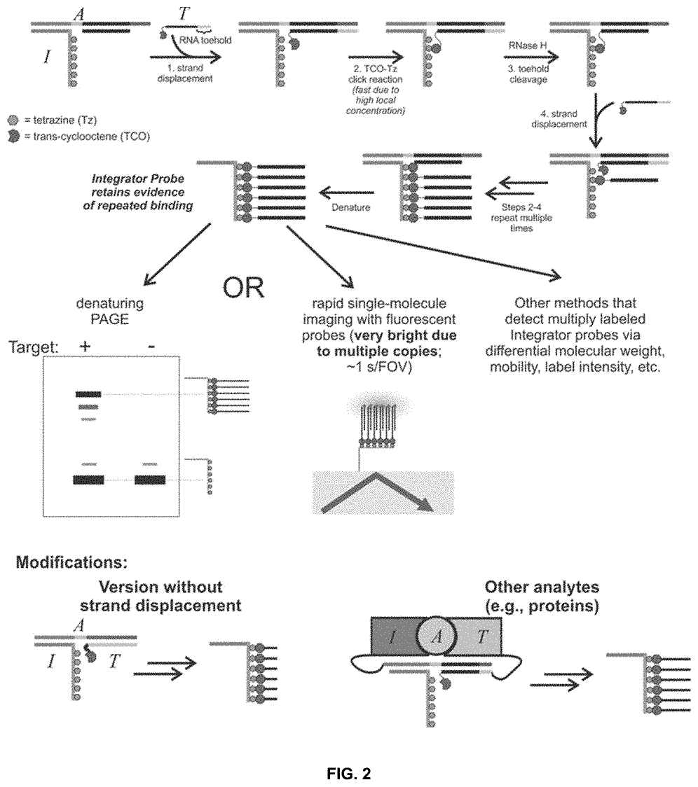

An exemplary embodiment of the technology for detecting a nucleic acid (e.g., DNA) analyte is shown in FIG. 2. In this exemplary embodiment, each integrator probe comprises multiple tetrazine moieties (Tz); each tally probe comprises a region of RNA nucleotides (“RNA toehold”) and one trans-cyclooctene moiety. A click chemistry reaction (e.g., a copper-free click chemistry reaction) between a tetrazine moiety (Tz) and a trans-cyclooctene moiety (TCO) is used to form a covalent bond between an integrator probe I and a tally probe T (e.g., comprising a label U. Hybridization mediates the interaction between tally T and analyte A. The interaction is rendered transient by the presence of RNase H, which degrades the portion of T that contains RNA nucleotides complementary to the analyte sequence. After multiple cycles of tally probe binding, click reaction, and degradation, multiple copies of T (which are partly degraded by RNase H) are covalently bound to each integrator probe. The analyte is detected by detecting integrator probe comprising multiple (e.g., more than 2) partially degraded tally probe molecules (e.g., comprising a label). For example, a multiply-bound integrator probe can be determined by denaturing polyacrylamide gel electrophoresis (PAGE) or by total internal reflection fluorescence (TIRF) microscopy (FIG. 2). This technology can be used with a relatively long or short interaction between the tally probe and the analyte and can be adapted for detection of other types of analytes, such as proteins (FIG. 2).

In some embodiments, the tally probe is a nucleic acid or an aptamer. In some embodiments, the tally probe is a low-affinity antibody, antibody fragment, or nanobody. In some embodiments, the tally probe is a DNA-binding protein, RNA-binding protein, or a DNA-binding ribonucleoprotein complex. In some embodiments, the tally probe comprises multiple types of molecules or moieties as described herein (e.g., nucleic acid and protein).

In some embodiments, the integrator probe is a high-affinity antibody, antibody fragment, or nanobody. In some embodiments, the integrator probe is a nucleic acid. In some embodiments, the integrator probe comprises multiple types of molecules or moieties as described herein (e.g., nucleic acid and protein). In some embodiments, the integrator probe comprises a separate phase of matter from the bulk mixture, such as a colloidal nanoparticle or a distinct phase of a liquid-in-liquid emulsion or other multi-phase system.

### Analytes

The technology is not limited in the analyte that is detected, quantified, identified, or otherwise characterized (e.g., presence, absence, amount, concentration, state). The term “analyte” as used herein is a broad term and is used in its ordinary sense, including, without limitation, to refer to a substance or chemical constituent in a sample such as a biological sample (e.g., a biofluid such as, e.g., blood, interstitial fluid, cerebral spinal fluid, lymph fluid or urine) that can be analyzed. Analytes can include naturally occurring substances, artificial substances, metabolites, and/or reaction products. In some embodiments, the analyte comprises a salt, sugars, protein, fat, vitamin, or a hormone. In some embodiments, the analyte is naturally present in a biological sample (e.g., is “endogenous”); for example, in some embodiments, the analyte is a metabolic product, a hormone, an antigen, an antibody, and the like. Alternatively, in some embodiments, the analyte is introduced into a biological organism (e.g., is “exogenous), for example, a drug, drug metabolite, a drug precursor (e.g., prodrug), a contrast agent for imaging, a radioisotope, a chemical agent, etc. The metabolic products of drugs and pharmaceutical compositions are also contemplated analytes.

In some embodiments, the analyte is a polypeptide, a nucleic acid, a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, an organism, etc. In some embodiments, the analyte comprises a polypeptide, a nucleic acid, a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, an organism, etc. In some embodiments, the analyte comprises a combination of one or more of a polypeptide, a nucleic acid, a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, an organism, etc.

In some embodiments, the analyte is a nucleic acid as described herein. In some embodiments, the analyte is a nucleic acid that comprises a mutation (e.g., a single nucleotide polymorphism, a deletion, an insertion, a rearrangement, a fusion, etc.) In some embodiments, the analyte is a protein as described herein. In some embodiments, the analyte is a protein that comprises an amino acid substitution.

In some embodiments, the analyte is part of a multimolecular complex, e.g., a multiprotein complex, a nucleic acid/protein complex, a molecular machine, an organelle (e.g., a cell-free mitochondrion, e.g., in plasma; a plastid; golgi, endoplasmic reticulum, vacuole, peroxisome, lysosome, and/or nucleus), cell, virus particle, tissue, organism, or any macromolecular complex or structure or other entity that is amenable to analysis by the technology described herein (e.g., a ribosome, spliceosome, vault, proteasome, DNA polymerase III holoenzyme, RNA polymerase II holoenzyme, symmetric viral capsids, GroEL/GroES; membrane protein complexes: photosystem I, ATP synthase, nucleosome, centriole and microtubule-organizing center (MTOC), cytoskeleton, flagellum, nucleolus, stress granule, germ cell granule, or neuronal transport granule). For example, in some embodiments a multimolecular complex comprises an analyte and the technology finds use in characterizing, identifying, quantifying, and/or detecting one or more molecules (analytes) associated with (e.g., that is a component of) the multimolecular complex. In some embodiments an extracellular vesicle is isolated and the technology finds use in characterizing, identifying, quantifying, and/or detecting one or more molecules (analytes) associated with the vesicle. In some embodiments, the technology finds use in characterizing, identifying, quantifying, and/or detecting a protein (e.g., a surface protein) and/or an analytes present inside the vesicle, e.g., a protein, nucleic acid, or other analyte described herein. In some embodiments, the vesicle is fixed and permeabilized prior to analysis.

In some embodiments, the interaction between the analyte and the tally probe is distinguishably influenced by a covalent modification of the analyte. For example, in some embodiments, the analyte is a polypeptide comprising a post-translational modification, e.g., a protein or a peptide comprising a post-translational modification. In some embodiments, a post-translational modification of a polypeptide affects the association of a tally probe with the analyte, e.g., the integrator probe signal is a function of the presence or absence of the post-translational modification on the polypeptide. In some embodiments, the interaction between the analyte comprising a post-translational modification and the tally probe is mediated by a chemical affinity tag, e.g., a chemical affinity tag comprising a nucleic acid. For example, in some embodiments, the analyte is a nucleic acid comprising an epigenetic modification, e.g., a nucleic acid comprising a methylated base. In some embodiments, a modification of a nucleic acid affects the binding of a tally probe with the analyte, e.g., the integrator probe signal is a function of the presence or absence of the modification on the nucleic acid. In some embodiments, the analyte is a nucleic acid comprising a covalent modification to a nucleobase, a ribose, or a deoxyribose moiety of the target analyte.

In some embodiments, the analyte is subjected to thermal denaturation in the presence of a carrier prior to providing an integrator probe and/or a tally probe. In some embodiments, the analyte is subjected to chemical denaturation in the presence of a carrier prior to providing an integrator probe and/or a tally probe, e.g., the analyte is denatured with a denaturant such as urea, formamide, guanidinium chloride, high ionic strength, low ionic strength, high pH, low pH, or sodium dodecyl sulfate (SDS).

As used herein “detect an analyte” or “detect a substance” will be understood to encompass direct detection of the analyte itself or indirect detection of the analyte by detecting its by-product(s).

### Fluorescent Labels and Fluorescent Moieties

In some embodiments, the technology comprises use of a fluorescent moiety (e.g., a fluorogenic dye, also referred to as a “fluorophore” or a “fluor”). For example, in some embodiments, an integrator probe comprises one or more fluorescent labels (e.g., comprising a fluorescent moiety). In some embodiments, a tally probe comprises a fluorescent label (e.g., comprising a fluorescent moiety). A wide variety of fluorescent moieties is known in the art and methods are known for linking a fluorescent moiety to a molecule (e.g., an integrator probe and/or a tally probe).

Examples of compounds that may be used as the fluorescent moiety include but are not limited to xanthene, anthracene, cyanine, porphyrin, and coumarin dyes. Examples of xanthene dyes that find use with the present technology include but are not limited to fluorescein, 6-carboxyfluorescein (6-FAM), 5-carboxyfluorescein (5-FAM), 5- or 6-carboxy-4, 7, 2′, 7′-tetrachlorofluorescein (TET), 5- or 6-carboxy-2′, 4, 4′, 5′, 7, 7′-hexachlorofluorescein (HEX), 5′ or 6′-carboxy-4′,5′-dichloro-2,7′-dimethoxyfluorescein (JOE), 5-carboxy-2′, 4′, 5′, 7′-tetrachlorofluorescein (ZOE), rhodol, rhodamine, tetramethylrhodamine (TAMRA), 4, 7-dichlorotetramethyl rhodamine (DTAMRA), rhodamine X (ROX), and Texas Red. Examples of cyanine dyes that may find use with the present invention include but are not limited to Cy 3, Cy 3B, Cy 3.5, Cy 5, Cy 5.5, Cy 7, and Cy 7.5. Other fluorescent moieties and/or dyes that find use with the present technology include but are not limited to energy transfer dyes, composite dyes, and other aromatic compounds that give fluorescent signals. In some embodiments, the fluorescent moiety comprises a quantum dot.

In some embodiments, the fluorescent moiety comprises a fluorescent protein (e.g., a green fluorescent protein (GFP), a modified derivative of GFP (e.g., a GFP comprising S65T, an enhanced GFP (e.g., comprising F64L)), or others known in the art such as, e.g., blue fluorescent protein (e.g., EBFP, EBFP2, Azurite, mKalamal), cyan fluorescent protein (e.g., ECFP, Cerulean, CyPet, mTurquoise2), and yellow fluorescent protein derivatives (e.g., YFP, Citrine, Venus, YPet). Embodiments provide that the fluorescent protein may be covalently or noncovalently bonded to one or more integrator and/or tally probes.

Fluorescent dyes include, without limitation, d-Rhodamine acceptor dyes including Cy 5, dichloro[R110], dichloro[R6G], dichloro[TAMRA], dichloro[ROX] or the like, fluorescein donor dyes including fluorescein, 6-FAM, 5-FAM, or the like; Acridine including Acridine orange, Acridine yellow, Proflavin, pH 7, or the like; Aromatic Hydrocarbons including 2-Methylbenzoxazole, Ethyl p-dimethylaminobenzoate, Phenol, Pyrrole, benzene, toluene, or the like; Arylmethine Dyes including Auramine O, Crystal violet, Crystal violet, glycerol, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dyes including 1,1′-diethyl-2,2′-cyanine iodide, Cryptocyanine, Indocarbocyanine (C3) dye, Indodicarbocyanine (C5) dye, Indotricarbocyanine (C7) dye, Oxacarbocyanine (C3) dye, Oxadicarbocyanine (C5) dye, Oxatricarbocyanine (C7) dye, Pinacyanol iodide, Stains all, Thiacarbocyanine (C3) dye, ethanol, Thiacarbocyanine (C3) dye, n-propanol, Thiadicarbocyanine (C5) dye, Thiatricarbocyanine (C7) dye, or the like; Dipyrrin dyes including N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, or the like; Merocyanines including 4-(dicyanomethylene)-2-methyl-6-(p-dimethylaminostyryl)-4H-pyran (DCM), acetonitrile, 4-(dicyanomethylene)-2-methyl-6-(p-dimethylaminostyryl)-4H-pyran (DCM), methanol, 4-Dimethylamino-4′-nitrostilbene, Merocyanine 540, or the like; Miscellaneous Dyes including 4′,6-Diamidino-2-phenylindole (DAPI), dimethylsulfoxide, 7-Benzylamino-4-nitrobenz-2-oxa-1,3-diazole, Dansyl glycine, Dansyl glycine, dioxane, Hoechst 33258, DMF, Hoechst 33258, Lucifer yellow CH, Piroxicam, Quinine sulfate, Quinine sulfate, Squarylium dye III, or the like; Oligophenylenes including 2,5-Diphenyloxazole (PPO), Biphenyl, POPOP, p-Quaterphenyl, p-Terphenyl, or the like; Oxazines including Cresyl violet perchlorate, Nile Blue, methanol, Nile Red, ethanol, Oxazine 1, Oxazine 170, or the like; Polycyclic Aromatic Hydrocarbons including 9,10-Bis(phenylethynyl)anthracene, 9,10-Diphenylanthracene, Anthracene, Naphthalene, Perylene, Pyrene, or the like; polyene/polyynes including 1,2-diphenylacetylene, 1,4-diphenylbutadiene, 1,4-diphenylbutadiyne, 1,6-Diphenylhexatriene, Beta-carotene, Stilbene, or the like; Redox-active Chromophores including Anthraquinone, Azobenzene, Benzoquinone, Ferrocene, Riboflavin, Tris(2,2′-bipyridypruthenium(II), Tetrapyrrole, Bilirubin, Chlorophyll a, diethyl ether, Chlorophyll a, methanol, Chlorophyll b, Diprotonated-tetraphenylporphyrin, Hematin, Magnesium octaethylporphyrin, Magnesium octaethylporphyrin (MgOEP), Magnesium phthalocyanine (MgPc), PrOH, Magnesium phthalocyanine (MgPc), pyridine, Magnesium tetramesitylporphyrin (MgTMP), Magnesium tetraphenylporphyrin (MgTPP), Octaethylporphyrin, Phthalocyanine (Pc), Porphin, ROX, TAMRA, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), pyridine, Zinc tetramesitylporphyrin (ZnTMP), Zinc tetramesitylporphyrin radical cation, Zinc tetraphenylporphyrin (ZnTPP), or the like; Xanthenes including Eosin Y, Fluorescein, basic ethanol, Fluorescein, ethanol, Rhodamine 123, Rhodamine 6G, Rhodamine B, Rose bengal, Sulforhodamine 101, or the like; or mixtures or combination thereof or synthetic derivatives thereof.

Several classes of fluorogenic dyes and specific compounds are known that are appropriate for particular embodiments of the technology: xanthene derivatives such as fluorescein, rhodamine, Oregon green, eosin, and Texas red; cyanine derivatives such as cyanine, indocarbocyanine, oxacarbocyanine, thiacarbocyanine, and merocyanine; naphthalene derivatives (dansyl and prodan derivatives); coumarin derivatives; oxadiazole derivatives such as pyridyloxazole, nitrobenzoxadiazole, and benzoxadiazole; pyrene derivatives such as cascade blue; oxazine derivatives such as Nile red, Nile blue, cresyl violet, and oxazine 170; acridine derivatives such as proflavin, acridine orange, and acridine yellow; arylmethine derivatives such as auramine, crystal violet, and malachite green; and tetrapyrrole derivatives such as porphin, phtalocyanine, bilirubin. In some embodiments the fluorescent moiety a dye that is xanthene, fluorescein, rhodamine, BODIPY, cyanine, coumarin, pyrene, phthalocyanine, phycobiliprotein, ALEXA FLUOR® 350, ALEXA FLUOR® 405, ALEXA FLUOR® 430, ALEXA FLUOR® 488, ALEXA FLUOR®514, ALEXA FLUOR® 532, ALEXA FLUOR®546, ALEXA FLUOR® 555, ALEXA FLUOR®568, ALEXA FLUOR® 568, ALEXA FLUOR®594, ALEXA FLUOR®610, ALEXA FLUOR®633, ALEXA FLUOR®647, ALEXA FLUOR®660, ALEXA FLUOR®680, ALEXA FLUOR® 700, ALEXA FLUOR® 750, or a squaraine dye. In some embodiments, the label is a fluorescently detectable moiety as described in, e.g., Haugland (September 2005) MOLECULAR PROBES HANDBOOK OF FLUORESCENT PROBES AND RESEARCH CHEMICALS (10th ed.), which is herein incorporated by reference in its entirety.

In some embodiments the label (e.g., a fluorescently detectable label) is one available from ATTO-TEC GmbH (Am Eichenhang 50, 57076 Siegen, Germany), e.g., as described in U.S. Pat. Appl. Pub. Nos. 20110223677, 20110190486, 20110172420, 20060179585, and 20030003486; and in U.S. Pat. No. 7,935,822, all of which are incorporated herein by reference (e.g., ATTO 390, ATO 425, ATTO 465, ATO 488, ATTO 495, ATO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO740).

One of ordinary skill in the art will recognize that dyes having emission maxima outside these ranges may be used as well. In some cases, dyes ranging between 500 nm to 700 nm have the advantage of being in the visible spectrum and can be detected using existing photomultiplier tubes. In some embodiments, the broad range of available dyes allows selection of dye sets that have emission wavelengths that are spread across the detection range. Detection systems capable of distinguishing many dyes are known in the art.

### Label Transfer

Embodiments of the technology comprise transfer (e.g., irreversible transfer (e.g., substantially irreversible transfer and/or effectively irreversible transfer)) of labels from a plurality of tally probes to an integrator probe. The technology is not limited in the compositions, molecules, linkers, non-covalent and/or covalent binding, chemical reactions, etc. that provide transfer of a label from a tally probe to an integrator probe when the tally probe and integrator probe are bound to an analyte molecule. The technology provides a tally probe comprising a label that is stably associated with the tally probe until the tally probe and an integrator probe are bound to an analyte. Upon association of the tally probe and integrator probe to an analyte, the label is transferred to the integrator probe and is stably associated with the integrator probe. Accordingly, in some embodiments, the technology provides: a tally probe comprising a label; and an integrator probe, wherein the affinity of the label for unbound tally probe is higher than the affinity of the label for any other molecule in the composition comprising the tally probe and the affinity of the label for the tally probe bound to an analyte is less than the affinity of the label for an integrator probe bound to the same analyte to which is bound the tally probe.

In some embodiments, a tally probe comprises a covalently linked label and a portion comprising RNA nucleotides that are complementary to a DNA analyte, the label chemically reacts (e.g., by a click chemistry reaction) with an integrator probe bound to the same analyte to which is bound the tally probe, and an RNase H enzyme degrades the portion of the tally probe comprising the RNA nucleotides to provide for binding of a different tally probe comprising a covalently linked label and a portion comprising RNA nucleotides that are complementary to a DNA analyte to provide a second cycle of label transfer to the integrator probe.

In some embodiments, an integrator probe comprises one or more nucleic acid duplexes comprising a toehold sequence, and a tally probe comprises a nucleic acid that is transferred to the integrator probe by toehold-mediated strand displacement.

In some embodiments, an integrator probe comprises a plurality of labels, each of which is in a first state and is converted to a second state by a tally probe bound to the same analyte to which is bound the integrator probe. Conversion of a plurality of integrator labels from the first state to the second state provides a record of the binding of a plurality of tally probes to the analyte.

In some embodiments, a tally probe comprises an antibody that binds a label and an integrator probe comprises a plurality of antibodies that each bind the label with a higher affinity than the antibody of the tally probe binds the label.

In some embodiments, an integrator probe comprises one or more enzymes that catalyze(s) the transfer of one or more labels to the integrator probe. In some embodiments, a tally probe comprises one or more enzymes that catalyze(s) the transfer of a label to the integrator probe.

In some embodiments, a tally probe interacts with the analyte indirectly via a separate adaptor probe (e.g., nucleic acid probe, aptamer, or antibody) that is bound stably to the analyte. Upon binding the adaptor probe, the tally probe transfers a label to an adjacent integrator probe associated to the same molecule of analyte, then dissociates from the adaptor probe. Many labels may be transferred to the same integrator probe from several tally probes that sequentially bind the same analyte via the same adaptor probe.

In some embodiments, binding of a tally probe to an analyte associated with a separate phase of matter in the reaction mixture (e.g., a colloidal particle, a lipid nanoparticle, a micelle, or a separate phase of a liquid-liquid emulsion, coacervate, or aqueous two-phase system) results in the transfer of a label to the separate phase of matter. The integrator comprises the separate phase of matter.

### Detection

In some embodiments, the technology comprises detecting an integrator probe comprising a plurality of labels (e.g., at least 2 labels). The technology provides for the detection of target analytes, e.g., in the presence of similar analytes and, in some embodiments, background noise. The technology is not limited in the detector or other detection technology that finds use in detecting the integrator probe comprising a plurality of labels. In some embodiments, the label is a fluorescent label and detecting an integrator probe comprising a plurality of labels comprises detecting a fluorescent signal indicating that an integrator probe comprises a plurality of labels (e.g., a fluorescent signal indicating the presence of an integrator probe comprising more than one fluorescent label). In some embodiments, the intensity of the fluorescence signal indicates the number of labels attached to an integrator probe. In some embodiments, a threshold is established (e.g., a cut-off value) for the fluorescence intensity that distinguishes between an integrator probe comprising no or one label and an integrator probe comprising two or more labels. In some embodiments, a threshold is established (e.g., a cut-off value) for the fluorescence intensity that distinguishes between an integrator probe indicating the absence of an analyte (e.g., comprising a number of labels less than a threshold cutoff value) and an integrator probe indicating the presence of an analyte (e.g., comprising a number of labels that is at least a threshold value). In some embodiments, experiments are conducted using controls to establish a threshold (e.g., a cut-off value) for the fluorescence intensity and/or number of labels that distinguishes between the absence of an analyte (e.g., an integrator probe comprising no or one label) and the presence of an analyte (e.g., an integrator probe comprising two or more labels).

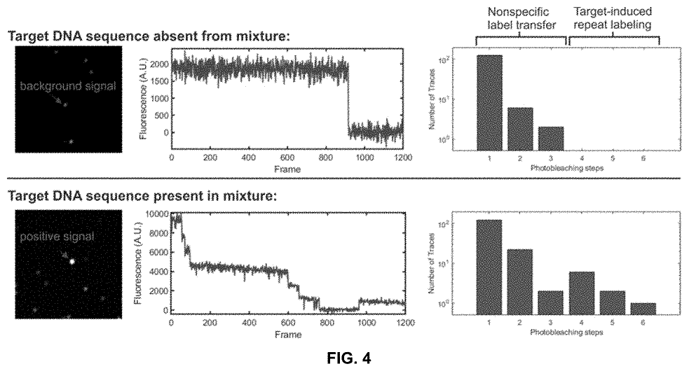

In some embodiments, a threshold number of labels is established indicating the presence from the absence of the analyte. For instance, in some embodiments, fewer than 2 labels (e.g., 0 or 1 label) indicates the absence of the analyte and 2 or more labels indicates the presence of the analyte. In some embodiments, fewer than 3 labels (e.g., 0, 1, or 2 labels) indicates the absence of the analyte and 3 or more labels indicates the presence of the analyte. In some embodiments, fewer than 4 labels (e.g., 0, 1, 2, or 3 labels) indicates the absence of the analyte and 4 or more labels indicates the presence of the analyte (see, e.g., Example 2 and FIG. 4). In some embodiments, fewer than 5 labels (e.g., 0, 1, 2, 3, or 4 labels) indicates the absence of the analyte and 5 or more labels indicates the presence of the analyte. In some embodiments, fewer than 6 labels indicates the absence of the analyte and 6 or more labels indicates the presence of the analyte. In some embodiments, fewer than 7 labels indicates the absence of the analyte and 7 or more labels indicates the presence of the analyte. In some embodiments, fewer than 8 labels indicates the absence of the analyte and 8 or more labels indicates the presence of the analyte. In some embodiments, fewer than 9 labels indicates the absence of the analyte and 9 or more labels indicates the presence of the analyte. In some embodiments, fewer than 10 labels indicates the absence of the analyte and 10 or more labels indicates the presence of the analyte. In some embodiments, thresholds are set at a cut-off of more than 10 labels.

In some embodiments, the appropriate threshold number of labels to detect the presence of the analyte is determined by statistical analysis of an appropriate blank or control measurement (e.g., by setting the threshold as 2, 3, 4, or 5 standard deviations above the mean or median number of labels per integrator detected, measured, and/or recorded for the blank or control measurement). In some embodiments, the appropriate threshold number of labels to detect the presence of the analyte is determined by subjecting the blank or control measurement to statistical analysis with a Poisson distribution, binomial distribution, normal distribution, or other applicable statistical model to determine (e.g., predict) the threshold number of labels that indicates the presence of the analyte with a probability of false positive detection events below a certain tolerable level (e.g., a probability of 0.01, 0.001, 0.0001, 0.00001, or 0.000001 that the detection of an analyte is a false positive).

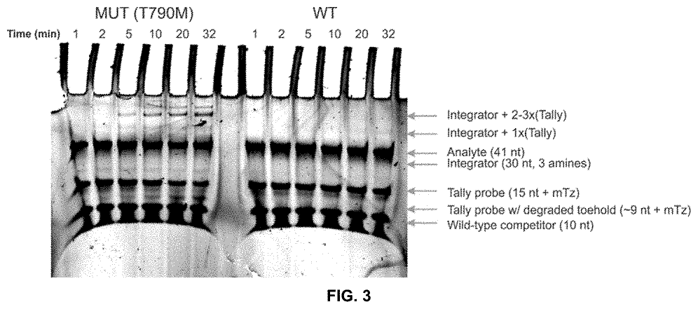

In some embodiments, the label changes the mass, charge, density, or hydrodynamic radius of an integrator probe and detecting an integrator probe comprising a plurality of labels comprises detecting a change in mass, charge, density, or hydrodynamic radius of an integrator probe indicating that an integrator probe comprises a plurality of labels (e.g., an assay comprising use of gel electrophoresis, mass spectrometry, gradient ultracentrifugation, chromatography, electrochemical assay). In some embodiments, detecting an integrator probe comprising a plurality of labels comprises detecting a change in mobility using an electrophoretic and/or chromatographic method. See, e.g., FIG. 2 and FIG. 3.

In some embodiments, a single-molecule detection method is used to detect an integrator probe comprising a plurality of labels. For example, in some embodiments, signal originating from an integrator probe comprising a plurality of labels is distinguishable from an integrator probe comprising zero labels or one label and/or from a tally probe comprising a label and/or from free label in a composition. In some embodiments, detecting an integrator probe comprising a plurality of labels comprises use of a technology such as, e.g., total internal reflection fluorescence (TIRF) or near-TIRF microscopy, zero-mode waveguides (ZMWs), light sheet microscopy, stimulated emission depletion (STED) microscopy, or confocal microscopy. In some embodiments, the integrator probe comprises a plurality fluorescent labels and the intensity of the fluorescence emission is proportional to the number of fluorescent labels attached to the integrator probe. In some embodiments, the intensity of the fluorescence emission from the integrator probe is compared to a known value of the intensity of the fluorescence emission for an integrator probe comprising one label. Detection of fluorescence having an intensity greater than the value for an integrator probe comprising one label indicates that the integrator probe comprises a plurality of labels and that the analyte is present in the sample. In some embodiments, the fluorescence of an integrator probe comprising a plurality of labels is greater than a defined threshold indicating the presence of the analyte in the sample. In some embodiments the fluorescence intensity of an integrator probe comprising a plurality of labels has a fluorescence intensity that is at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more standard deviations above the fluorescence intensity of an integrator probe comprising zero or one label or above a defined threshold indicating the presence of an analyte in a sample.

In some embodiments, the number of labels bound to an integrator probe is inferred by counting the number of drops in fluorescent intensity as the integrator probe is exposed to excitation light (e.g., by counting of photobleaching steps). In some embodiments, an integrator probe comprising a plurality of labels exhibits at least 2, 3, 4, 5, 6, 7, 8, 9, 10, 11, 12, 13, 14, 15, 16, 17, 18, 19, or 20 photobleaching steps, indicating the presence of an analyte in a sample. In some embodiments, an integrator probe exhibiting a number of photobleaching steps greater than that observed for integrator probes in control experiments in the absence of the analyte indicates the presence of an analyte in a sample.

In some embodiments, positive and/or negative control samples are measured (e.g., a control sample known to comprise or not to comprise an analyte). In embodiments comprising use of a fluorescent label, fluorescence detected in a negative control sample is “background fluorescence” or “background (fluorescence) intensity” or “baseline”.

In some embodiments, data are analyzed by algorithms that recognize patterns and regularities in data, e.g., using artificial intelligence, pattern recognition, machine learning, statistical inference, neural nets, etc. In some embodiments, the analysis comprises use of a frequentist analysis and in some embodiments the analysis comprises use of a Bayesian analysis. In some embodiments, pattern recognition systems are trained using known “training” data (e.g., using supervised learning) and in some embodiments algorithms are used to discover previously unknown patterns (e.g., unsupervised learning). See, e.g., Duda, et al. (2001) Pattern classification (2nd edition), Wiley, New York; Bishop (2006) Pattern Recognition and Machine Learning, Springer. In some embodiments, pattern recognition (e.g., using training sets, supervised learning, unsupervised learning, and analysis of unknown samples) associates identified patterns with analytes such that particular patterns provide a “fingerprint” of particular analytes that find use in detection, quantification, and identification of analytes. In some embodiments, the data are treated using statistics to determine the probability of an analyte being present or absent in a sample.

### Samples

In some embodiments, a sample (e.g., a biological sample and/or a biofluid) comprises an analyte. In some embodiments, the sample also comprises a variety of other non-analyte components, such as nucleic acids, proteins, lipids, and metabolites. Analytes and samples comprising analytes can be obtained from any material (e.g., cellular material (live or dead), extracellular material, viral material, environmental samples (e.g., metagenomic samples), and/or synthetic material), obtained from an animal, plant, bacterium, archaeon, fungus, or any other organism. Biological samples for use in the present technology include viral particles or preparations thereof. Analytes and samples comprising analytes can be obtained directly from an organism or from a biological sample obtained from an organism, e.g., from blood, urine, cerebrospinal fluid, seminal fluid, saliva, sputum, stool, hair, sweat, tears, skin, and tissue. Exemplary samples include, but are not limited to, whole blood, lymphatic fluid, serum, plasma, buccal cells, sweat, tears, saliva, sputum, hair, skin, biopsy, cerebrospinal fluid (CSF), amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone marrow, fine needle, etc.), washes (e.g., oral, nasopharyngeal, bronchial, bronchialalveolar, optic, rectal, intestinal, vaginal, epidermal, etc.), and/or other specimens.

Any tissue or body fluid specimen may be used as a source for analytes and samples comprising analytes for use in the technology, including forensic specimens, archived specimens, preserved specimens, and/or specimens stored for long periods of time, e.g., fresh-frozen, methanol/acetic acid fixed, or formalin-fixed paraffin embedded (FFPE) specimens and samples. Analytes and samples comprising analytes can also be isolated from cultured cells, such as a primary cell culture or a cell line. The cells or tissues from which analytes and samples comprising analytes are obtained can be infected with a virus or other intracellular pathogen.

Nucleic acid molecules can be obtained, e.g., by extraction from a biological sample, e.g., by a variety of techniques such as those described by Maniatis, et al. (1982) Molecular Cloning: A Laboratory Manual, Cold Spring Harbor, N.Y. (see, e.g., pp. 280-281). A sample can comprise RNA extracted from a biological specimen, a cDNA library, viral nucleic acid, or genomic DNA. A sample may also be DNA isolated from a non-cellular origin, e.g. amplified/isolated DNA that has been stored in a freezer.

In some embodiments, the technology is used to identify an analyte in situ. In particular, embodiments of the technology provide for the identification of an analyte directly in a tissue, cell, etc. (e.g., after permeabilizing the tissue, cell, etc.) without extracting the analyte from the tissue, cell, etc. In some embodiments of the technology related to in situ detection, the technology is applied in vivo, ex vivo, and/or in vitro. In some embodiments, the sample is a crude sample, a minimally treated cell lysates, or a biofluid lysate. In some embodiments, the analyte is detected in a crude lysates without nucleic acid purification.

### Methods

Some embodiments provide a method of identifying an analyte using an integrator probe and a tally probe as described herein. For example, in some embodiments, methods comprise obtaining or providing a sample (e.g., a biological sample (e.g., a biofluid)), e.g., a sample comprising an analyte and/or suspected to comprise an analyte. In some embodiments, the sample is obtained from and/or provided by a patient in need of testing for the presence, absence, and/or quantity of an analyte. In some embodiments, testing a sample provided by and/or obtained from a patient for the presence, absence, and/or quantity of an analyte indicates the status (e.g., the presence, absence, and/or quantity) of the analyte in the patient. In some embodiments, a sample is subjected to preliminary processing designed to isolate or enrich the sample for the analyte. A variety of techniques known to those of ordinary skill in the art may be used for this purpose, including but not limited to centrifugation, immunocapture, cell lysis, filtration, chromatography, use of magnetic beads, and nucleic acid target capture.

In some embodiments, methods comprise providing an integrator probe and providing a tally probe. In some embodiments, methods comprise mixing a composition comprising an analyte with one or more compositions comprising an integrator probe and/or a tally probe. In some embodiments, methods comprise contacting a composition comprising an analyte with an integrator probe and a plurality of tally probes to provide a reaction mixture and incubating the reaction mixture at an incubation temperature for an incubation time.

In some embodiments, providing a tally probe comprises synthesizing a tally probe. In some embodiments, synthesizing a tally probe comprises synthesizing the tally probe and attaching (e.g., non-covalently or covalently attaching) a label to the tally probe. In some embodiments, methods comprise providing the label. In some embodiments, the label is provided by a commercial source and, in some embodiments, the label is synthesized by a user of the methods provided herein. In some embodiments, a labeled tally probe is provided by a commercial source. In some embodiments, providing an integrator probe comprises synthesizing an integrator probe.

In some embodiments, methods comprise incubating a composition comprising a tally probe and an integrator probe (e.g., at a temperature that is an incubation temperature and/or for a length of time that is an incubation time). In some embodiments, methods comprise incubating a composition comprising a tally probe and an integrator probe for a length of time at the incubation temperature so that multiple cycles of tally probe binding to an analyte, if present, occur to produce an integrator probe comprising multiple labels. In some embodiments, methods comprise detecting an integrator probe comprising multiple labels. In some embodiments, methods comprise quantifying the number of integrator probes comprising multiple labels and/or quantifying the number of labels (e.g., label moieties) transferred from a tally probe to one or more integrator probes. In some embodiments, methods comprise quantifying an analyte in a sample by quantifying the number of integrator probes comprising multiple labels and/or quantifying the number of labels (e.g., label moieties) transferred from a tally probe to an integrator probe. In some embodiments, methods comprise producing a standard curve by quantifying the number of integrator probes comprising multiple labels and/or quantifying the number of labels (e.g., label moieties) transferred from a tally probe to an integrator probe in one or more compositions comprising a known amount and/or concentration of analyte under known conditions (e.g., a known incubation temperature and/or a known incubation time). In some embodiments, methods comprise providing a negative control and/or providing a positive control. In some embodiments, methods comprise performing an assay method with a negative control and/or with a positive control, e.g., by providing composition comprising the negative control and/or positive control, a tally probe, and an integrator probe.

In some embodiments, methods comprise detecting a signal from a label. In some embodiments, methods comprise providing a source of electromagnetic radiation. In some embodiments, methods comprise providing a detector of electromagnetic radiation. In some embodiments, methods comprise irradiating a sample (e.g., using a source of electromagnetic radiation). In some embodiments, methods comprise quantifying an intensity of electromagnetic radiation, e.g., emitted by a label, using a detector of electromagnetic radiation. In some embodiments, methods comprise calculating a concentration or amount of an analyte in a sample using an intensity of a signal produced by a label (e.g., by one or more labeled integrator probes).

In some embodiments, methods comprise providing environmental conditions (e.g., temperature, solution conditions (e.g., pH, buffering, salt, chemical activities)), catalysts, and/or reactive chemical components so that a label is transferred from a tally probe to an integrator probe when a tally probe and an integrator probe are bound to the same analyte. Furthermore, in some embodiments, methods comprise providing environmental conditions (e.g., temperature, solution conditions (e.g., pH, buffering, salt, chemical activities)), catalysts, and/or reactive chemical components so that a tally probe comprising a label binds to an analyte to which is bound an integrator probe, a label is transferred from the tally probe to the integrator probe, and the tally probe dissociates from the analyte. In some embodiments, methods comprise providing an enzymatic activity that promotes dissociation of a tally probe from an analyte after label transfer.

### Kits

In some embodiments, the technology relates to kits. For example, in some embodiments, kits comprise a tally probe comprising a label and an integrator probe, e.g., in one or more compositions (e.g., a composition comprising the tally probe comprising a label and the integrator probe; a first composition comprising the tally probe comprising a label and a second composition comprising an integrator probe). In some embodiments, kits comprise a positive control (e.g., a composition comprising a known concentration and/or amount of an analyte). In some embodiments, kits comprise a negative control (e.g., a composition comprising no analyte or an undetectable amount and/or concentration of an analyte). In some embodiments, kits comprise a buffer solution for preparing a composition comprising an analyte. In some embodiments, kits comprise a buffer solution and a device for preparing a sample from a patient. In some embodiments, kits comprise a vessel for containing a sample from a subject and in which an assay as described herein is performed.

### Systems

Embodiments of the technology relate to systems for detecting analytes. For example, in some embodiments, the technology provides a system for quantifying one or more target analytes, wherein the system comprises an integrator probe and a tally probe comprising a label as described herein. Furthermore, some system embodiments comprise a detection component that records a signal from the integrator probe after incubation of the integrator probe and tally probe with a sample comprising an analyte, if present. For example, in some embodiments the detection component records a signal produced from the integrator probe, e.g., after interaction of the integrator probe and tally probe with an analyte. In some embodiments, the detection component records the intensity of a signal provided by an integrator probe comprising a plurality of labels.

System embodiments comprise analytical processes (e.g., embodied in a set of instructions, e.g., encoded in software, that direct a microprocessor to perform the analytical processes) to process a signal (e.g., from an integrator probe comprising a plurality of labels) and to identify a sample as a sample comprising an analyte. In some embodiments, analytical processes use the intensity of the signal produced by an integrator probe comprising a plurality of labels as input data. In some embodiments, systems comprise an analyte. Embodiments of systems are not limited in the analyte that is detected. For example, in some embodiments the analyte is polypeptide, e.g., a protein or a peptide. In some embodiments, the target analyte is a nucleic acid. In some embodiments, the target analyte is a small molecule or other molecule or entity as described herein.

Some system embodiments of the technology comprise components for the detection and quantification of an analyte. Some system embodiments comprise a detection component that is a fluorescence microscope comprising an illumination configuration to excite labels of integrator probes. Some embodiments comprise a fluorescence detector, e.g., a detector comprising an intensified charge coupled device (ICCD), an electron-multiplying charge coupled device (EM-CCD), a complementary metal-oxide-semiconductor (CMOS), a photomultiplier tube (PMT), an avalanche photodiode (APD), and/or another detector capable of detecting fluorescence emission from single chromophores. Some particular embodiments comprise a component configured for lens-free imaging, e.g., a lens-free microscope, e.g., a detection and/or imaging component for directly imaging on a detector (e.g., a CMOS) without using a lens.

Some embodiments comprise a computer and software encoding instructions for the computer to perform, e.g., to control data acquisition and/or analytical processes for processing data.

Some embodiments comprise optics, such as lenses, mirrors, dichroic mirrors, optical filters, etc., e.g., to detect fluorescence selectively within a specific range of wavelengths or multiple ranges of wavelengths.

For example, in some embodiments, computer-based analysis software is used to translate the raw data generated by the detection assay (e.g., the presence, absence, or amount of one or more analytes) into data of predictive value for a clinician. The clinician can access the predictive data using any suitable means.

Some system embodiments comprise a computer system upon which embodiments of the present technology may be implemented. In various embodiments, a computer system includes a bus or other communication mechanism for communicating information and a processor coupled with the bus for processing information. In various embodiments, the computer system includes a memory, which can be a random access memory (RAM) or other dynamic storage device, coupled to the bus, and instructions to be executed by the processor. Memory also can be used for storing temporary variables or other intermediate information during execution of instructions to be executed by the processor. In various embodiments, the computer system can further include a read only memory (ROM) or other static storage device coupled to the bus for storing static information and instructions for the processor. A storage device, such as a magnetic disk or optical disk, can be provided and coupled to the bus for storing information and instructions.

In various embodiments, the computer system is coupled via the bus to a display, such as a cathode ray tube (CRT) or a liquid crystal display (LCD), for displaying information to a computer user. An input device, including alphanumeric and other keys, can be coupled to the bus for communicating information and command selections to the processor. Another type of user input device is a cursor control, such as a mouse, a trackball, or cursor direction keys for communicating direction information and command selections to the processor and for controlling cursor movement on the display. This input device typically has two degrees of freedom in two axes, a first axis (e.g., x) and a second axis (e.g., y), that allows the device to specify positions in a plane.

A computer system can perform embodiments of the present technology. Consistent with certain implementations of the present technology, results can be provided by the computer system in response to the processor executing one or more sequences of one or more instructions contained in the memory. Such instructions can be read into the memory from another computer-readable medium, such as a storage device. Execution of the sequences of instructions contained in the memory can cause the processor to perform the methods described herein. Alternatively, hard-wired circuitry can be used in place of or in combination with software instructions to implement the present teachings. Thus, implementations of the present technology are not limited to any specific combination of hardware circuitry and software.

The term “computer-readable medium” as used herein refers to any medium that participates in providing instructions to the processor for execution. Such a medium can take many forms, including but not limited to, non-volatile media, volatile media, and transmission media. Examples of non-volatile media can include, but are not limited to, optical or magnetic disks, such as a storage device. Examples of volatile media can include, but are not limited to, dynamic memory. Examples of transmission media can include, but are not limited to, coaxial cables, copper wire, and fiber optics, including the wires that comprise the bus.

Common forms of computer-readable media include, for example, a floppy disk, a flexible disk, hard disk, flash medium, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

Various forms of computer readable media can be involved in carrying one or more sequences of one or more instructions to the processor for execution. For example, the instructions can initially be carried on the magnetic disk of a remote computer. The remote computer can load the instructions into its dynamic memory and send the instructions over a network connection (e.g., a LAN, a WAN, the internet, a telephone line). A local computer system can receive the data and transmit it to the bus. The bus can carry the data to the memory, from which the processor retrieves and executes the instructions. The instructions received by the memory may optionally be stored on a storage device either before or after execution by the processor.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

In accordance with such a computer system, some embodiments of the technology provided herein further comprise functionalities for collecting, storing, and/or analyzing data (e.g., presence, absence, concentration of an analyte). For example, some embodiments contemplate a system that comprises a processor, a memory, and/or a database for, e.g., storing and executing instructions, analyzing label signals, signal intensities, and/or detection data, performing calculations using the data, transforming the data, and storing the data. In some embodiments, an algorithm applies a statistical model to the data.

Many diagnostics involve determining the presence of, or a nucleotide sequence of, one or more nucleic acids.

In some embodiments, an equation comprising variables representing the presence, absence, concentration, amount, or sequence properties of one or more analytes produces a value that finds use in making a diagnosis or assessing the presence or qualities of an analyte. As such, in some embodiments this value is presented by a device, e.g., by an indicator related to the result (e.g., an LED, an icon on a display, a sound, or the like). In some embodiments, a device stores the value, transmits the value, or uses the value for additional calculations. In some embodiments, an equation comprises variables representing the presence, absence, concentration, amount, or properties of one or more analytes.

Thus, in some embodiments, the present technology provides the further benefit that a clinician, who is not likely to be trained in analytical assays, need not understand the raw data. The data are presented directly to the clinician in its most useful form. In some embodiments, the clinician is then able to utilize the information to optimize the care of a subject. The present technology contemplates any method capable of receiving, processing, and transmitting the information to and from laboratories conducting the assays, information providers, medical personal, and/or subjects. For example, in some embodiments of the present technology, a sample is obtained from a subject and submitted to a profiling service (e.g., a clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using electronic communication systems). Once received by the profiling service, the sample is processed, and a profile is produced that is specific for the diagnostic or prognostic information desired for the subject. The profile data are then prepared in a format suitable for interpretation by a treating clinician. For example, rather than providing raw data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor. In some embodiments, the information is first analyzed at the point of care or at a regional facility. The raw data are then sent to a central processing facility for further analysis and/or to convert the raw data to information useful for a clinician or patient. The central processing facility provides the advantage of privacy (all data are stored in a central facility with uniform security protocols), speed, and uniformity of data analysis. The central processing facility can then control the fate of the data following treatment of the subject. For example, using an electronic communication system, the central facility can provide data to the clinician, the subject, or researchers. In some embodiments, the subject is able to access the data using the electronic communication system. The subject may choose further intervention or counseling based on the results. In some embodiments, the data are used for research use. For example, the data may be used to further optimize the inclusion or elimination of markers as useful indicators of a particular condition associated with the disease.

### Uses

The technology is not limited in its use. For example, the technology finds use in research for detecting the presence of an analyte and/or for quantifying an analyte. In some embodiments, the technology finds use in clinical medicine, e.g., in detecting an analyte that indicates that a patient has a disease, malady, and/or ailment.

Various embodiments relate to the detection of a wide range of analytes. For example, in some embodiments the technology finds use in detecting a nucleic acid (e.g., a DNA or RNA). In some embodiments, the technology finds use in detecting a nucleic acid comprising a particular target sequence. In some embodiments, the technology finds use in detecting a nucleic acid comprising a particular mutation (e.g., a single nucleotide polymorphism, an insertion, a deletion, a missense mutation, a nonsense mutation, a genetic rearrangement, a gene fusion, etc.). In some embodiments, the technology finds use in detection a polypeptide (e.g., a protein, a peptide). In some embodiments, the technology finds use in detecting a polypeptide encoded by a nucleic acid comprising a mutation (e.g., a polypeptide comprising a substitution, a truncated polypeptide, a mutant or variant polypeptide).

In some embodiments, the technology finds use in detecting post-translational modifications to polypeptides (e.g., phosphorylation, methylation, acetylation, glycosylation (e.g., O-linked glycosylation, N-linked glycosylation, ubiquitination, attachment of a functional group (e.g., myristoylation, palmitoylation, isoprenylation, prenylation, farnesylation, geranylation, geranylgeranylation, glypiation, glycosylphosphatidylinositol (GPI) anchor formation), hydroxylation, biotinylation, pegylation, oxidation, SUMOylation, disulfide bridge formation, disulfide bridge cleavage, proteolytic cleavage, amidation, sulfation, pyrrolidone carboxylic acid formation. In some embodiments, the technology finds use in the detection of the loss of these features, e.g., dephosporylation, demethylation, deacetylation, deglycosylation, deamidation, dehydroxylation, deubiquitination, etc. In some embodiments, the technology finds use in detecting epigenetic modifications to DNA or RNA (e.g., methylation (e.g., methylation of CpG sites), hydroxymethylation). In some embodiments, the technology finds use in detecting the loss of these features, e.g., demethylation of DNA or RNA, etc. In some embodiments, the technology finds use in detecting alterations in chromatin structure, nucleosome structure, histone modification, etc., and in detecting damage to nucleic acids. In some embodiments, the technology finds use in detecting a lipid, carbohydrate, metabolite, and/or small molecule.

In some embodiments, the technology finds use as a molecular diagnostic assay, e.g., to assay samples having small specimen volumes (e.g., a droplet of blood, e.g., for mail-in service). In some embodiments, the technology provides for the early detection of cancer or infectious disease using sensitive detection of very low-abundance analyte biomarkers. In some embodiments, the technology finds use in molecular diagnostics to assay epigenetic modifications of protein biomarkers (e.g., post-translational modifications).

In some embodiments, the technology finds use in characterizing multimolecular complexes (e.g., characterizing one or more components of a multimolecular complex), e.g., a multiprotein complex, a nucleic acid/protein complex, a molecular machine, an organelle (e.g., a cell-free mitochondrion, e.g., in plasma), cell, virus particle, organism, tissue, or any macromolecular structure or entity that is amenable to analysis by the technology described herein. For example, in some embodiments a multimolecular complex is isolated and the technology finds use in characterizing, identifying, quantifying, and/or detecting one or more molecules (analytes) associated with the multimolecular complex. In some embodiments, an extracellular vesicle is isolated and the technology finds use in characterizing, identifying, quantifying, and/or detecting one or more molecules (analytes) associated with the vesicle. In some embodiments, the technology finds use in characterizing, identifying, quantifying, and/or detecting a protein (e.g., a surface protein) and/or an analyte present inside the vesicle, e.g., a protein, nucleic acid, or other analyte described herein. In some embodiments, the vesicle is fixed and permeabilized prior to analysis.

### EXAMPLES

### Example 1

During the development of embodiments of the technology described herein, experiments were conducted to detect a point mutation in a nucleic acid using an embodiment of the integrator technology described herein. In the experiment, an integrator probe comprising one or more (e.g., 1, 2, or 3) trans-cyclooctene (TCO) moieties and a tally probe comprising methyltetrazine (mTz) were used to detect a cytosine-to-thymine point mutation in a nucleic acid encoding epidermal growth factor receptor (EGFR). This mutation produces a substitution of methionine for threonine at position 790 in the amino acid sequence of the EGFR protein (EGFR T790M).

In an exemplary experiment, the EGFR T790M nucleic acid (“MUT”) was 41 bp in length. The tally probe comprised 10 consecutive nucleotides complementary to the portion of MUT containing the single-nucleotide mutation. Six nucleotides of the tally probe 10-nt complementary sequence were RNA nucleotides and provided a region that was targeted for degradation by RNase H upon binding of the tally probe to the MUT nucleic acid. A wild-type competitor probe was provided that comprised 10 DNA nucleotides that are complementary to the wild-type (WT) EGFR sequence. The competitor probe competed with the tally probe for binding to the wild-type sequence and suppressed nonspecific signal that could be generated by interaction of the tally probe with the wild-type nucleic acid. The analyte (MUT nucleic acid), integrator probe, tally probe, and competitor probe were incubated with RNase H in NEBuffer 3.1 (New England Biolabs) at room temperature for 1-30 minutes before quenching the click reaction with approximately 2 mM methyltetrazine. Reactions were run on a denaturing polyacrylamide gel and SYBR gold staining was used to visualize nucleic acids within the gel (FIG. 3).

As described above, the tally probe was designed to form 10 base pairs with the nucleic acid encoding EGFR T790M and a competitor probe was designed to form 10 base pairs with the wild-type nucleotide sequence to suppress binding of the tally probe to the wild-type nucleic acid. Accordingly, the experiment was designed so that the integrator TCO moieties reacted (e.g., by click chemistry reactions) with multiple copies of the tally probe only if the mutant sequence EGFR T790M was present and not if the wild-type sequence was present. Data collected during the experiment were consistent with this expectation. As shown in FIG. 3, low-mobility bands appeared in a denaturing polyacrylamide gel electrophoresis experiment upon incubation of the integrator probe, tally probe, wild-type competitor probe, and RNase H with the mutant nucleic acid (MUT). These low-mobility bands indicated the presence of the integrator probe conjugated to 2-3 copies of the tally probe. In contrast, there was no evidence of a multiply-modified integrator probe in the presence of only the wild-type sequence (FIG. 3), demonstrating the ability of the technology described herein to distinguish analytes, e.g., nucleic acids comprising sequences that differ by as little as a single nucleotide.

### Example 2

During the development of embodiments of the technology provided herein, experiments were conducted in which the technology was used to detect an analyte DNA sequence encoding the EGFR T790M mutation using a single integrator molecule and single-molecule total internal reflection fluorescence (TIRF) microscopy. Samples comprised a target analyte that was a DNA comprising a nucleotide sequence encoding the EGFR T790M mutation (FIG. 4, bottom) or lacked the target analyte (negative control; FIG. 4, top). After incubation of the negative control with an integrator probe specific for the sequence encoding the EGFR mutation, a tally probe, and RNase H, the integrator probes typically comprised only 0, 1, or 2 fluorescent labels and were not observed to comprise more than 3 fluorescent labels (FIG. 4, top). In contrast, after incubation of a sample comprising the target analyte with the integrator probe, the tally probes, and RNase H, a significant subset of integrator probes comprised 4, 5, or 6 labels, indicating the presence of the target sequence in the mixture (FIG. 4, bottom). The number of labels associated with each integrator probe after incubation can be inferred by the number of drops in fluorescence (e.g., corresponding to photobleaching steps) observed for each surface-bound integrator probe as the sample is exposed to excitation light. In the particular system used in these experiments, the integrator probe was configured to bind to as many as six labels, which comprise a DNA sequence, and the labels were detected by sequence-specific hybridization of a fluorescently labeled DNA probe to the label. Furthermore, these data indicate that embodiments of the technology provided herein detect single integrator molecules (e.g., using TIRF microscopy) that were bound to single analyte molecules during the incubation period. As shown in FIG. 4, multiply labeled integrator probes appear as brighter puncta in the microscope image and can be detected by counting the number of photobleaching steps per integrator molecule.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A composition for detecting an analyte in a sample, said composition comprising:
an integrator probe that is capable of stably associating with an analyte; and
a tally probe comprising a label and that is capable of directly or indirectly associating with said analyte,
wherein irreversible transfer of said label from said tally probe to said integrator probe occurs more rapidly and/or more efficiently when said tally probe and said integrator probe are both associated with said analyte than when said tally probe and/or said integrator probe is/are dissociated from said analyte.

2. The composition of claim 1 further comprising an adaptor probe that is capable of binding to said analyte and wherein said tally probe indirectly associates with said analyte by directly associating with said adaptor probe.

3. The composition of claim 1 wherein said tally probe is specific for said analyte and directly associates with said analyte.

4. The composition of claim 1 wherein said integrator probe is specific for said analyte.

5. The composition of claim 1 wherein said tally probe transiently binds said analyte.

6. The composition of claim 2 wherein said tally probe transiently binds said adaptor probe.

7. The composition of claim 1 wherein said integrator probe is configured to bind more than one label.

8. The composition of claim 1 comprising an integrator probe comprising multiple labels.

9-11. (canceled)

12. The composition of claim 1 further comprising an analyte comprising a nucleic acid, a protein, a metabolite, a small molecule, a lipid, or a sugar.

13. The composition of claim 1 wherein said integrator probe comprises a separate phase of matter.

14. The composition of claim 13 wherein said separate phase of matter is a colloidal particle, liposome, micelle, or emulsified droplet.

15. The composition of claim 1 wherein said integrator probe comprises a nucleic acid or a protein.

16. The composition of claim 1 wherein said tally probe comprises a nucleic acid or a protein.

17. (canceled)

18. The composition of claim 1 further comprising a tally probe release component that converts a tally probe bound to an analyte to a dissociated tally probe.

19. The composition of claim 18 wherein said tally probe release component comprises an enzyme specific for a tally probe-analyte complex.

20. The composition of claim 1 wherein said integrator probe comprises a first functional group and said label of said tally probe comprises a second functional group that is reactive with the first functional group.

21. The composition of claim 1 wherein said integrator probe comprises a first click reactant and said label of said tally probe comprises a second click reactant.

22. A system for detecting an analyte in a sample, said system comprising:
an integrator probe that is capable of stably associating with an analyte; and
a tally probe comprising a label and that is capable of directly or indirectly associating with said analyte,
wherein irreversible transfer of said label from said tally probe to said integrator probe occurs more rapidly and/or more efficiently when said tally probe and said integrator probe are both associated with said analyte than when said tally probe and/or said integrator probe is/are dissociated from said analyte; and
a detection component configured to detect and/or quantify a signal produced by an integrator probe comprising multiple labels.

23-43. (canceled)

44. A method of detecting and/or quantifying an analyte, said method comprising:
providing a sample comprising an analyte;
providing an integrator probe that is capable of stably associating with an analyte; and
providing a tally probe comprising a label and that is capable of directly or indirectly associating with said analyte.

45-73. (canceled)
